SEQUENCE LISTING (142 sequences)

SEQ ID NO: 1 - protein, 5 aa, synthetic construct
SYAMS

SEQ ID NO: 2 - protein, 17 aa, synthetic construct
AISGSGGSTYYADSVKG

SEQ ID NO: 3 - protein, 11 aa, synthetic construct
PTSRYSYGYDY

SEQ ID NO: 4 - protein, 11 aa, synthetic construct
PTSRYSYSYNN

SEQ ID NO: 5 - protein, 5 aa, synthetic construct
SHAMS

SEQ ID NO: 6 - protein, 7 aa, synthetic construct
VDTKFDP

SEQ ID NO: 7 - protein, 5 aa, synthetic construct
SYPMS

SEQ ID NO: 8 - protein, 17 aa, synthetic construct
TISTGGTYTYYPDSVKG

SEQ ID NO: 9 - protein, 15 aa, synthetic construct
QGPIYYGNYYYAMDY

SEQ ID NO: 10 - protein, 17 aa, synthetic construct
TISTGGRYTYYPDSVKG

SEQ ID NO: 11 - protein, 15 aa, synthetic construct
QGPILRKNYYYGMDV

SEQ ID NO: 12 - protein, 15 aa, synthetic construct
QGPIYYGNYYYGMDV

SEQ ID NO: 13 - protein, 17 aa, synthetic construct
TISTGGSHTYYPDSVKG

SEQ ID NO: 14 - protein, 16 aa, synthetic construct
HGYLLYDGYYEWYFDV

SEQ ID NO: 15 - protein, 17 aa, synthetic construct
TISTGGSHTYYPESVKG

SEQ ID NO: 16 - protein, 16 aa, synthetic construct
HGYLLYEGYYEWYFDY

SEQ ID NO: 17 - protein, 5 aa, synthetic construct
GYPMS

SEQ ID NO: 18 - protein, 17 aa, synthetic construct
TISTGARHTYYPDSVKG

SEQ ID NO: 19 - protein, 15 aa, synthetic construct
HEGLRRGKYHCIMDY

SEQ ID NO: 20 - protein, 15 aa, synthetic construct
HEGLRRGKYHSIMDY

SEQ ID NO: 21 - protein, 13 aa, synthetic construct
TGNSNNVGDQGAV

SEQ ID NO: 22 - protein, 7 aa, synthetic construct
RNNNRPS

SEQ ID NO: 23 - protein, 11 aa, synthetic construct
STWDSGLNSVV

SEQ ID NO: 24 - protein, 11 aa, synthetic construct
STWDKNNKSVV

SEQ ID NO: 25 - protein, 13 aa, synthetic construct
SGSSSNIGSNTVN

SEQ ID NO: 26 - protein, 7 aa, synthetic construct
SNNQRPS

SEQ ID NO: 27 - protein, 11 aa, synthetic construct
EVWDDSLNGPV

SEQ ID NO: 28 - protein, 16 aa, synthetic construct
RSSQSLVHSDGNTYLH

SEQ ID NO: 29 - protein, 7 aa, synthetic construct
KVSNRFS

SEQ ID NO: 30 - protein, 9 aa, synthetic construct
SQSTHVPYT

SEQ ID NO: 31 - protein, 16 aa, synthetic construct
RSSQSLVDGEDGTYLN

SEQ ID NO: 32 - protein, 7 aa, synthetic construct
KVSERFD

SEQ ID NO: 33 - protein, 16 aa, synthetic construct
RSSQSLVDGQDGTYLH

SEQ ID NO: 34 - protein, 7 aa, synthetic construct
KVSNRFD

SEQ ID NO: 35 - protein, 16 aa, synthetic construct
RSSQSLVNQEGETYLH

SEQ ID NO: 36 - protein, 15 aa, synthetic construct
KASQSVDYDGHSFLN

SEQ ID NO: 37 - protein, 7 aa, synthetic construct
AASNLES

SEQ ID NO: 38 - protein, 10 aa, synthetic construct
QQSTEDPPYT

SEQ ID NO: 39 - protein, 15 aa, synthetic construct
RASESVDYDGHSFIN

SEQ ID NO: 40 - protein, 7 aa, synthetic construct
AASNKDT

SEQ ID NO: 41 - protein, 15 aa, synthetic construct
RASQSVDYDGHSFLN

SEQ ID NO: 42 - protein, 7 aa, synthetic construct
AASNLQS

SEQ ID NO: 43 - protein, 15 aa, synthetic construct
KSSQSVDYDGHSFLN

SEQ ID NO: 44 - protein, 7 aa, synthetic construct
AASNRES

SEQ ID NO: 45 - protein, 15 aa, synthetic construct
RASQSVDYEGHSFLN

SEQ ID NO: 46 - protein, 10 aa, synthetic construct
QQSTENPPYT

SEQ ID NO: 47 - protein, 15 aa, synthetic construct
KSSQSVDYEGHSFLN

SEQ ID NO: 48 - protein, 15 aa, synthetic construct
KASQSIDYDGDSFLN

SEQ ID NO: 49 - protein, 9 aa, synthetic construct
HQCNEDPYM

SEQ ID NO: 50 - protein, 15 aa, synthetic construct
RASESVDYDGDSFIN

SEQ ID NO: 51 - protein, 9 aa, synthetic construct
HQSNEDPYM

SEQ ID NO: 52 - protein, 9 aa, synthetic construct
HQSNEDPYL

SEQ ID NO: 53 - protein, 9 aa, synthetic construct
HQSNEDPYV

SEQ ID NO: 54 - protein, 15 aa, synthetic construct
RASQSIDYDGDSFLN

SEQ ID NO: 55 - protein, 9 aa, synthetic construct
QQSNEDPYV

SEQ ID NO: 56 - protein, 15 aa, synthetic construct
KSSQSIDYDGDSFLN

SEQ ID NO: 57 - protein, 120 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPTSRYSYGYDYWGQGTLVTVSS

SEQ ID NO: 58 - protein, 120 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPTSRYSYSYNNWGQGTLVTVSS

SEQ ID NO: 59 - protein, 116 aa, synthetic construct
EVQLVQSGGGVVQPGRSLRLSCAASGFIFSSHAMSWVRQAPGKGLEWVSAISGSGGSTYYADSVKGRFTISRDNSKNTLYLQMNSLRAEDTAVYYCAKVDTKFDPWGQGTLVTVSS

SEQ ID NO: 60 - protein, 124 aa, synthetic construct
EVKLVESGGGLVKPGGSLKLSCAASGFTFSSYPMSWVRQTPEKRLEWVATISTGGTYTYYPDSVKGRFTISRDNAKNTLYLQMSSLRSEDTAMYYCARQGPIYYGNYYYAMDYWGQGTSVTVSS

SEQ ID NO: 61 - protein, 124 aa, synthetic construct
EVQLVESGGGLVKPGGSLRLSCAASGFTFSSYPMSWVRQAPGKGLEWVSTISTGGTYTYYPDSVKGRFTISRDNAKNSLYLQMNSLRAEDTAVYYCARQGPIYYGNYYYAMDYWGQGTTVTVSS

SEQ ID NO: 62 - protein, 124 aa, synthetic construct
EVQLVESGGGLVKPGGSLRLSCAASGFTFSSYPMSWVRQAPEKGLEWVSTISTGGRYTYYPDSVKGRFTISRDNAKNSLYLQMNSLRAEDTAVYYCARQGPIYYGNYYYAMDYWGQGTTVTVSS

SEQ ID NO: 63 - protein, 124 aa, synthetic construct
EVQLVESGGGLVKPGGSLRLSCAASGFTFSSYPMSWVRQAPEKGLEWVSTISTGGTYTYYPDSVKGRFTISRDNAKNSLYLQMNSLRAEDTAVYYCARQGPILRKNYYYGMDVWGQGTTVTVSS

SEQ ID NO: 64 - protein, 124 aa, synthetic construct
EVQLVESGGGLVKPGGSLRLSCAASGFTFSSYPMSWVRQAPEKGLEWVSTISTGGTYTYYPDSVKGRFTISRDNAKNSLYLQMNSLRAEDTAVYYCARQGPIYYGNYYYGMDVWGQGTTVTVSS

SEQ ID NO: 65 - protein, 125 aa, synthetic construct
EVQLQESGGGLVKPGGSLKLSCAASGFTFNSYAMSWVRQTPEKRLEWVATISTGGSHTYYPDSVKGRFTISRDNAKNTLYLQMSSLRSEDTAMYYCARHGYLLYDGYYEWYFDVWGAGTTVTVSS

SEQ ID NO: 66 - protein, 125 aa, synthetic construct
EVQLVESGGGLVKPGGSLRLSCAASGFTFNSYAMSWVRQAPGKGLEWVSTISTGGSHTYYPDSVKGRFTISRDNAKNSLYLQMNSLRAEDTAVYYCARHGYLLYDGYYEWYFDYWGQGTLVTVSS

SEQ ID NO: 67 - protein, 125 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSS

SEQ ID NO: 68 - protein, 124 aa, synthetic construct
EVKLMESGGDLVKPGGSLKLSCVASGFTFSGYPMSWVRQTPEKRLEWVATISTGARHTYYPDSVKGRFTISRDNAKNTLYLQMSSLRSEDTAMYYCARHEGLRRGKYHCIMDYWGQGTSVTVSS

SEQ ID NO: 69 - protein, 124 aa, synthetic construct
EVQLVESGGGLVKPGGSLRLSCAASGFTFSGYPMSWVRQAPGKGLEWVSTISTGARHTYYPDSVKGRFTISRDNAKNSLYLQMNSLRAEDTAVYYCARHEGLRRGKYHSIMDYWGQGTLVTVSS

SEQ ID NO: 70 - protein, 110 aa, synthetic construct
QAGLTQPPSVSKGLRQTATLTCTGNSNNVGDQGAVWLQQHQGHPPRLLSYRNNNRPSGISERFSASRSGNTASLTITGLQPEDEADYYCSTWDSGLNSVVFGGGTQLTVL

SEQ ID NO: 71 - protein, 110 aa, synthetic construct
QAVLTQPPSASGTPGQRVTISCTGNSNNVGDQGAVWYQQLPGTAPKLLIYRNNNRPSGVPDRFSGSKSGTSASLAISGLQSEDEADYYCSTWDKNNKSVVFGGGTKLTVL

SEQ ID NO: 72 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLRSEDEADYYCEVWDDSLNGPVFGGGTKVTVL

SEQ ID NO: 73 - protein, 112 aa, synthetic construct
DIVMTQPPLSLPVSLGDQASISCRSSQSLVHSDGNTYLHWYLQRPGQSPKLLIYKVSNRFSGVPDRFSGSGSGTDFTLKISRVEAEDLGVYFCSQSTHVPYTFGGGTKLEIK

SEQ ID NO: 74 - protein, 112 aa, synthetic construct
DIVMTQSPLSLPVTLGQPASISCRSSQSLVHSDGNTYLHWYQQRPGQSPKLLIYKVSNRFSGVPDRFSGSGSGTDFTLKISRVEAEDVGVYYCSQSTHVPYTFGGGTKVEIK

SEQ ID NO: 75 - protein, 112 aa, synthetic construct
DIVMTQSPLSLPVTLGDPASISCRSSQSLVDGEDGTYLNWFQQRPGQSPKLLIYKVSERFDGVPDRFSGSGSGTDFTLKISRVEAEDVGVYYCSQSTHVPYTFGGGTKVEIK

SEQ ID NO: 76 - protein, 112 aa, synthetic construct
DVVMTQSPLSLPVTLGDPASISCRSSQSLVDGQDGTYLHWYQQRPGQSPKLLIYKVSNRFDGVPDRFSGSGSGTDFTLKISRVEAEDVGVYYCSQSTHVPYTFGQGTKLEIK

SEQ ID NO: 77 - protein, 112 aa, synthetic construct
DVVMTQSPLSLPVTLGDPASISCRSSQSLVNQEGETYLHWYQQRPGQSPKLLIYKVSNRFDGVPDRFSGSGSGTDFTLKISRVEAEDVGVYYCSQSTHVPYTFGQGTKLEIK

SEQ ID NO: 78 - protein, 112 aa, synthetic construct
DIVLTQSPASLAVSLGQRATISCKASQSVDYDGHSFLNWYQQKPGQPPKLLIYAASNLESGLPARFSGSGSGTDFTLNIHPVEEEDAATYYCQQSTEDPPYTFGGGTKLEIK

SEQ ID NO: 79 - protein, 112 aa, synthetic construct
DIVLTQSPASLAVSPGQRATITCRASESVDYDGHSFINWYQQKPGQPPKLLIYAASNKDTGVPARFSGSGSGTDFTLTINPVEAEDTANYYCQQSTEDPPYTFGQGTKLEIK

SEQ ID NO: 80 - protein, 112 aa, synthetic construct
DIQMTQSPSSLSASVGDRVTITCRASQSVDYDGHSFLNWYQQKPGKAPKLLIYAASNLQSGVPSRFSGSGSGTDFTLTISSLQPEDFATYYCQQSTEDPPYTFGQGTKLEIK

SEQ ID NO: 81 - protein, 112 aa, synthetic construct
DIVMTQSPDSLAVSLGERATINCKSSQSVDYDGHSFLNWYQQKPGQPPKLLIYAASNRESGVPDRFSGSGSGTDFTLTISSLQAEDVAVYYCQQSTEDPPYTFGQGTKLEIK

SEQ ID NO: 82 - protein, 112 aa, synthetic construct
DIQMTQSPSSLSASVGDRVTITCRASQSVDYEGHSFLNWYQQKPGKAPKLLIYAASNLQSGVPSRFSGSGSGTDFTLTISSLQPEDFATYYCQQSTENPPYTFGQGTKLEIK

SEQ ID NO: 83 - protein, 112 aa, synthetic construct
DIVMTQSPDSLAVSLGERATINCKSSQSVDYEGHSFLNWYQQKPGQPPKLLIYAASNRESGVPDRFSGSGSGTDFTLTISSLQAEDVAVYYCQQSTENPPYTFGQGTKLEIK

SEQ ID NO: 84 - protein, 111 aa, synthetic construct
DILLTQSPASLAVSLGQRATISCKASQSIDYDGDSFLNWYQQKPGQPPKLLIYAASNLESGIPARFSGSGSGTDFTLNIHPVEEEDAATYYCHQCNEDPYMFGGGTKLEIR

SEQ ID NO: 85 - protein, 111 aa, synthetic construct
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYMFGQGTKLEIK

SEQ ID NO: 86 - protein, 111 aa, synthetic construct
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYLFGQGTKLEIK

SEQ ID NO: 87 - protein, 111 aa, synthetic construct
DIVLTQSPASLAVSPGQRATITCRASESVDYDGDSFINWYQQKPGQPPKLLIYAASNKDTGVPARFSGSGSGTDFTLTINPVEAEDTANYYCHQSNEDPYVFGQGTKLEIK

SEQ ID NO: 88 - protein, 111 aa, synthetic construct
DIQMTQSPSSLSASVGDRVTITCRASQSIDYDGDSFLNWYQQKPGKAPKLLIYAASNLQSGVPSRFSGSGSGTDFTLTISSLQPEDFATYYCQQSNEDPYVFGQGTKLEIK

SEQ ID NO: 89 - protein, 111 aa, synthetic construct
DIVMTQSPDSLAVSLGERATINCKSSQSIDYDGDSFLNWYQQKPGQPPKLLIYAASNRESGVPDRFSGSGSGTDFTLTISSLQAEDVAVYYCQQSNEDPYVFGQGTKLEIK

SEQ ID NO: 90 - protein, 136 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 91 - protein, 115 aa, synthetic construct
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 92 - protein, 136 aa, synthetic construct
IPPHVQQSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 93 - protein, 136 aa, synthetic construct
IPPHVQKSVNNDMIVTDNNGAVKFPQLCKFCDVSFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 94 - protein, 136 aa, synthetic construct
IPPHVQKSVNNDMIVTDNNGAVKFPQLCKFCDVHFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 95 - protein, 136 aa, synthetic construct
IPPHVQKSVNNDMIVTDNNGAVKFPQLCKFCDVRFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 96 - protein, 136 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKESSKPGETFFMCSCSSDECNDNIIFSEEYNTSNPD

SEQ ID NO: 97 - protein, 136 aa, synthetic construct
IPPHVQQSVNNDMIVTDNNGAVKFPQLCKFCDVSFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 98 - protein, 136 aa, synthetic construct
IPPHVQQSVNNDMIVTDNNGAVKFPQLCKFCDVRFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 99 - protein, 136 aa, synthetic construct
IPPHVQQSVNNDMIVTDNNGAVKFPQLCKFCDVRFSTCDNQKSCMSNCSITSICEKPQEVCVAVWRKNDENITLETVCHDPKLPYHDFILEDAASPKCIMKESSKPGETFFMCSCSSDECNDNIIFSEEYNTSNPD

SEQ ID NO: 100 - protein, 136 aa, synthetic construct
IPPHVQQSVNNDMIVTDNNGAVKFPQLCKFCDVSFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 101 - protein, 136 aa, synthetic construct
IPPHVQQSVNNDMIVTDNNGAVKFPQLCKFCDVRFSTCDNQKSCMSNCSITSICEKPQEVCVAVWSSNDENITLETVCHDPKLPYHDFILEDAASPKCIMKESSKPGETFFMCSCSSDECNDNIIFSEEYNTSNPD

SEQ ID NO: 102 - protein, 136 aa, synthetic construct
IPPHVQQSVNNDMIVTDNNGAVKFPQLCKFCDVSFSTCDNQKSCMSNCSITSICEKPQEVCVAVWSSNDENITLETVCHDPKLPYHDFILEDAASPKCIMKESSKPGETFFMCSCSSDECNDNIIFSEEYNTSNPD

SEQ ID NO: 103 - protein, 129 aa, synthetic construct
SVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 104 - protein, 132 aa, synthetic construct
VQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 105 - protein, 127 aa, synthetic construct
NNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 106 - protein, 123 aa, synthetic construct
IVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 107 - protein, 119 aa, synthetic construct
NNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 108 - protein, 22 aa, synthetic construct
SGGGGSGGGGSGGGGSGGGGSG

SEQ ID NO: 109 - protein, 17 aa, synthetic construct
SGGGGSGGGGSGGGGSG

SEQ ID NO: 110 - protein, 12 aa, synthetic construct
SGGGGSGGGGSG

SEQ ID NO: 111 - protein, 7 aa, synthetic construct
SGGGGSG

SEQ ID NO: 112 - protein, 24 aa, synthetic construct
SGEAAAKEAAAKEAAAKEAAAKSG

SEQ ID NO: 113 - protein, 19 aa, synthetic construct
SGEAAAKEAAAKEAAAKSG

SEQ ID NO: 114 - protein, 14 aa, synthetic construct
SGEAAAKEAAAKSG

SEQ ID NO: 115 - protein, 22 aa, synthetic construct
SGEGESGEGESGEGESGEGESG

SEQ ID NO: 116 - protein, 20 aa, synthetic construct
EAAAKEAAAKEAAAKEAAAK

SEQ ID NO: 117 - protein, 21 aa, synthetic construct
GGGGSGGGGSGGGGSGGGGSG

SEQ ID NO: 118 - protein, 610 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGASGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 119 - protein, 612 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGASGEAAAKEAAAKEAAAKEAAAKSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 120 - protein, 610 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGASGEGESGEGESGEGESGEGESGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 121 - protein, 608 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAEAAAKEAAAKEAAAKEAAAKIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 122 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 123 - protein, 588 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 124 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 125 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVSFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 126 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVHFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 127 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 128 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKESSKPGETFFMCSCSSDECNDNIIFSEEYNTSNPD

SEQ ID NO: 129 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVSFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 130 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVRFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 131 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVRFSTCDNQKSCMSNCSITSICEKPQEVCVAVWRKNDENITLETVCHDPKLPYHDFILEDAASPKCIMKESSKPGETFFMCSCSSDECNDNIIFSEEYNTSNPD

SEQ ID NO: 132 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVSFSTCDNQKSCMSNCSITSICEKPQEVCVAVWSSNDENITLETVCHDPKLPYHDFILEDAASPKCIMKEKKKPGETFFMCSCSSDECNDNIIFSEEYNTSNPD

SEQ ID NO: 133 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVRFSTCDNQKSCMSNCSITSICEKPQEVCVAVWSSNDENITLETVCHDPKLPYHDFILEDAASPKCIMKESSKPGETFFMCSCSSDECNDNIIFSEEYNTSNPD

SEQ ID NO: 134 - protein, 609 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIPPHVQQSVNNDMIVTDNNGAVKFPQLCKFCDVSFSTCDNQKSCMSNCSITSICEKPQEVCVAVWSSNDENITLETVCHDPKLPYHDFILEDAASPKCIMKESSKPGETFFMCSCSSDECNDNIIFSEEYNTSNPD

SEQ ID NO: 135 - protein, 602 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 136 - protein, 605 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 137 - protein, 600 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 138 - protein, 596 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 139 - protein, 592 aa, synthetic construct
EVQLVESGGGLVKPGGSLRLSCAASGFTFSSYAMSWVRQAPGKGLEWVSTISTGGSHTYYPESVKGRFTISRDNAKNSLYLQMNSLRAEDTAVYYCARHGYLLYEGYYEWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 140 - protein, 16 aa, synthetic construct
HGYLLYDGYYEWYFDY

SEQ ID NO: 141 - protein, 219 aa, synthetic construct
DIVMTQSPLSLPVTLGDPASISCRSSQSLVDGEDGTYLNWFQQRPGQSPKLLIYKVSERFDGVPDRFSGSGSGTDFTLKISRVEAEDVGVYYCSQSTHVPYTFGGGTKVEIKRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 142 - protein, 606 aa, synthetic construct
EVQLVESGGGLVKPGGSLRLSCAASGFTFSSYPMSWVRQAPEKGLEWVSTISTGGRYTYYPDSVKGRFTISRDNAKNSLYLQMNSLRAEDTAVYYCARQGPIYYGNYYYAMDYWGQGTTVTVSSASKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

(19) United States
(12) Patent Application Publication
(10) Pub. No.: US 2024/0391990 A1
(43) Pub. Date: Nov. 28, 2024

(54) BI-FUNCTIONAL FUSION PROTEIN AND USES THEREOF

(71) Applicants: Nan TANG, Beijing (CN); PULMONGENE (HONG KONG) CO., LIMITED, Hong Kong (CN)

(72) Inventors: Nan TANG, Beijing (CN); Jianhua SUI, Beijing (CN); Ximing LIU, Beijing (CN); Nanmeng SONG, Beijing (CN); Rui ZHAO, Beijing (CN)

(21) Appl. No.: 18/688,572

(22) PCT Filed: Sep. 2, 2022
(86) PCT No.: PCT/CN2022/116919

§ 371 Date: Mar. 1, 2024

(30) Foreign Application Priority Data
Sep. 3, 2021 (WO) PCT/CN2021/116558

(51) Int. Cl. C07K 16/22; A61K 38/00; A61K 39/00; A61P 11/00; C07K 14/71

(52) U.S. Cl. CPC C07K 16/22; A61P 11/00; C07K 14/71; A61K 38/00; A61K 2039/505; C07K 2317/33; C07K 2317/565; C07K 2317/76; C07K 2317/92; C07K 2319/30; C07K 2319/32

(57) ABSTRACT

Provided is a bi-functional fusion protein that targets both TGFβ ligands and AREG, and blocks TGFβ and AREG signaling simultaneously, as well as a nucleic acid molecule encoding the bi-functional fusion protein, an expression vector for producing the bi-functional fusion protein, a host cell for producing the bi-functional fusion protein, and a method for preparing and/or characterizing the bi-functional fusion protein.

### CROSS-REFERENCE TO RELATED APPLICATIONS

This application is the national stage entry of International Patent Application No. PCT/CN2022/116919, filed on Sep. 2, 2022, and published as WO 2023/030511 A1 on Mar. 9, 2023, which claims priority to Chinese Patent Application No. PCT/CN2021/116558, filed on Sep. 3, 2021, which are hereby incorporated by reference herein in their entireties.

### BACKGROUND

### Introduction

Transforming growth factor beta (TGFβ) is a pleiotropic cytokine that plays an essential role under physiological and pathological conditions. TGFβ isoforms, namely β1, δ2, and δ3, are homodimeric polypeptides around 25 kDa. These ligands signal through cell surface receptors, including transforming growth factor R receptor I, II, and III (TRI, TRII, TRIII), and intracellular SMAD effector proteins such as SMAD 2 and 3. The transduction of the signal affects a series of cellular processes including cell survival, proliferation, differentiation, cell motility, and extracellular matrix production (ECM).

TGFβ is considered as a central regulator for fibrogenesis. It was shown that TGFβ 1 induces fibrosis in multiple organs through activation of myofibroblasts, production of excessive ECM components, and inhibition of ECM degradation. Blocking TGFβ signaling prevents and inhibits abnormal remodeling and scarring in a myriad of organs, including lung, liver, and kidney. TGFβ is also shown to play a key role in the immune system and is considered as one of the most potent immune suppressors for both innate and adaptive immune responses. Additionally, TGFβ is reported to exhibit protumor activities in some cancers through directly acting on tumor cells and/or tumor environment.

Anti-TGFβ therapies have been developed for fibrosis, certain cancers, and other diseases. TGFβ inhibitors include antisense oligonucleotides, small molecules inhibiting the kinase activities of the receptors, monoclonal antibodies directed against TGFβ ligands or receptors, and bifunctional proteins engineered with TGFβ traps. Specifically, TGFβ traps involve the engineering of TGFβ receptor through artificial dimerization of the edcodomains of these receptors.

Amphiregulin (AREG) is a low-affinity ligand in the epidermal growth factor (EGF) family. AREG protein is synthesized from a 252 amino acid transmembrane precursor, which is subjected to proteolytic cleavage within its ectodomain by cell membrane proteases, mainly TACE (tumor necrosis factor-α-converting enzyme). Mature soluble AREG then activates downstream signaling by binding directly to epidermal growth factor receptor (EGFR). This can elicit major intracellular signaling cascades including MAPK/ERK signaling to govern cell survival, proliferation, and motility.

AREG is shown to be specifically upregulated in alveolar type II cells (AT2s) in lung fibrosis models and patients with idiopathic pulmonary fibrosis (IPF). It is also demonstrated that AREG is both necessary and sufficient for the development of pulmonary fibrosis. Specifically, reducing the expression levels of AREG significantly attenuates the development of pulmonary fibrosis in a progressive lung fibrosis model. Overexpression of AREG in AT2s in mice induces remodeling and fibrotic changes in the lungs. Additionally, it was reported that the expression level of AREG is up-regulated in liver and kidney fibrosis, and that AREG is required for the development of fibrosis in liver, kidney, and skin. Therefore, AREG, as a profibrotic factor, is an attractive target not only for pulmonary fibrosis, but also for fibrosis in other organs.

AREG-EGFR signaling also plays a role in the immune system and during tumorigenesis. AREG is shown to be expressed at various immune cells under inflammatory conditions. The presence of AREG in various immune cell types and the activation pattern of these immune cells suggest that immune-derived AREG is associated with type 2 immune-mediated (Th2) resistance and tolerance mechanisms. Additionally, AREG is upregulated in a variety of cancers. Functional studies demonstrate that AREG can serve as a pro-oncogene in some cancers. These findings together suggest that targeting AREG activity can be a new therapeutic approach for chronic inflammation-associated diseases and cancers.

However, so far, no therapeutic approaches, including combination of two separate agents that harbor anti-TGFβ and anti-AREG activities, or, delivering a single bi-functional protein that possesses the capacity of inhibiting TGFβ and AREG concurrently, for treating the pathologies underlying the aforementioned diseases, including fibrosis, cancers, and diseases associated with chronic inflammation, have been proposed and verified.

### SUMMARY

### Summary of the Invention

The present invention provides a bi-functional fusion protein that targets both TGFβ ligands and AREG, and blocks TGFβ and AREG signaling simultaneously. The bi-functional fusion protein is an ideal candidate for treating fibrotic diseases, cancers, and diseases associated with chronic inflammation, including but not limited to renal fibrosis, hepatic fibrosis, and pulmonary fibrosis, in particular IPF. The present application also provides a nucleic acid molecule encoding the bi-functional fusion protein, an expression vector for producing the bi-functional fusion protein, a host cell for producing the bi-functional fusion protein, and a method for preparing and/or characterizing the bi-functional fusion protein. The present invention also provides use of the bi-functional fusion protein in the treatment, prevention and/or diagnosis of diseases, such as fibrotic diseases, cancers, and diseases associated with chronic inflammation, including but not limited to renal fibrosis, hepatic fibrosis, and pulmonary fibrosis, in particular IPF.

In the first aspect, the present invention provides a bi-functional fusion protein which comprises at least two domains that are capable of binding to AREG or a fragment thereof, and/or capable of binding to a TGFβ ligand or a fragment thereof.

In some embodiments, the bi-functional fusion protein comprises the first domain and the second domain, wherein the first domain is capable of binding to AREG or a fragment thereof, and the second domain is capable of sequestering a TGFβ ligand or a fragment thereof.

In some embodiments, the first domain is an antibody or an antigen-binding fragment thereof that binds to AREG or a fragment thereof, and the second domain is at least a part of the ectodomain of TGFβ receptor II (TGFβRII, TRII) or a variant thereof.

In some embodiments, the antibody or an antigen-binding fragment thereof is the anti-AREG antibody or fragment thereof, which is capable of binding to both human AREG (hAREG) and mouse AREG (mAREG).

In some embodiments, the anti-AREG antibody or fragment thereof according to the present invention is a human anti-AREG antibody, a murine anti-AREG antibody, a chimeric anti-AREG antibody, or a humanized anti-AREG antibody.

In some embodiments, the anti-AREG antibody or fragment thereof according to the present invention is capable of binding to a soluble form of AREG. Preferably, the anti-AREG antibody is capable of binding to the EGF-like domain of the soluble form of AREG.

In some embodiments, the anti-AREG antibody or fragment thereof according to the present invention is a single-chain antibody, a disulfied-linked Fv, DART, a diabody, a fragment comprising either a VL or VH domain. The fragment may be IgG, Fab, Fab′, F(ab′)₂, Fv, or scFv. The fragment also includes any synthetic or genetically engineered protein comprising an immunoglobulin variable region that acts like an antibody by binding to a specific antigen to form a complex. Regardless of structure, an antibody fragment binds with the same antigen that is recognized by the intact antibody.

In some embodiments, the anti-AREG antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, and HCDR3 are selected from a group consisting of: (1) HCDR1 shown by SEQ ID NO: 1, HCDR2 shown by SEQ ID NO: 2, HCDR3 shown by SEQ ID NO: 3; (2) HCDR1 shown by SEQ ID NO: 1, HCDR2 shown by SEQ ID NO: 2, HCDR3 shown by SEQ ID NO: 4; (3) HCDR1 shown by SEQ ID NO: 5, HCDR2 shown by SEQ ID NO: 2, HCDR3 shown by SEQ ID NO: 6; (4) HCDR1 shown by SEQ ID NO: 7, HCDR2 shown by SEQ ID NO: 8, HCDR3 shown by SEQ ID NO: 9; (5) HCDR1 shown by SEQ ID NO: 7, HCDR2 shown by SEQ ID NO: 10, HCDR3 shown by SEQ ID NO: 9; (6) HCDR1 shown by SEQ ID NO: 7, HCDR2 shown by SEQ ID NO: 8, HCDR3 shown by SEQ ID NO: 11; (7) HCDR1 shown by SEQ ID NO: 7, HCDR2 shown by SEQ ID NO: 8, HCDR3 shown by SEQ ID NO: 12; (8) HCDR1 shown by SEQ ID NO: 1, HCDR2 shown by SEQ ID NO: 13, HCDR3 shown by SEQ ID NO: 14; (9) HCDR1 shown by SEQ ID NO: 1, HCDR2 shown by SEQ ID NO: 13, HCDR3 shown by SEQ ID NO: 140; (10) HCDR1 shown by SEQ ID NO: 1, HCDR2 shown by SEQ ID NO: 15, HCDR3 shown by SEQ ID NO: 16; (11) HCDR1 shown by SEQ ID NO: 17, HCDR2 shown by SEQ ID NO: 18, HCDR3 shown by SEQ ID NO: 19; (12) HCDR1 shown by SEQ ID NO: 17, HCDR2 shown by SEQ ID NO: 18, HCDR3 shown by SEQ ID NO: 20; and (13) HCDR1, HCDR2, HCDR3 as shown in (1)-(12), but at least one of which includes one, two, three, four, or five amino acids addition, deletion, conservative amino acid substitution, or the combinations thereof, and LCDR1, LCDR2, and LCDR3 are selected from a group consisting of:
(1) LCDR1 shown by SEQ ID NO: 21, LCDR2 shown by SEQ ID NO: 22, LCDR3 shown by SEQ ID NO: 23; (2) LCDR1 shown by SEQ ID NO: 21, LCDR2 shown by SEQ ID NO: 22, LCDR3 shown by SEQ ID NO: 24; (3) LCDR1 shown by SEQ ID NO: 25, LCDR2 shown by SEQ ID NO: 26, LCDR3 shown by SEQ ID NO: 27; (4) LCDR1 shown by SEQ ID NO: 28, LCDR2 shown by SEQ ID NO: 29, LCDR3 shown by SEQ ID NO: 30; (5) LCDR1 shown by SEQ ID NO: 31, LCDR2 shown by SEQ ID NO: 32, LCDR3 shown by SEQ ID NO: 30; (6) LCDR1 shown by SEQ ID NO: 33, LCDR2 shown by SEQ ID NO: 34, LCDR3 shown by SEQ ID NO: 30; (7) LCDR1 shown by SEQ ID NO: 35, LCDR2 shown by SEQ ID NO: 34, LCDR3 shown by SEQ ID NO: 30; (8) LCDR1 shown by SEQ ID NO: 36, LCDR2 shown by SEQ ID NO: 37, LCDR3 shown by SEQ ID NO: 38; (9) LCDR1 shown by SEQ ID NO: 39, LCDR2 shown by SEQ ID NO: 40, LCDR3 shown by SEQ ID NO: 38; (10) LCDR1 shown by SEQ ID NO: 41, LCDR2 shown by SEQ ID NO: 42, LCDR3 shown by SEQ ID NO: 38; (11) LCDR1 shown by SEQ ID NO: 43, LCDR2 shown by SEQ ID NO: 44, LCDR3 shown by SEQ ID NO: 38; (12) LCDR1 shown by SEQ ID NO: 39, LCDR2 shown by SEQ ID NO: 40, LCDR3 shown by SEQ ID NO: 38; (13) LCDR1 shown by SEQ ID NO: 45, LCDR2 shown by SEQ ID NO: 42, LCDR3 shown by SEQ ID NO: 46; (14) LCDR1 SEQ ID NO: 47, LCDR2 shown by SEQ ID NO: 44, LCDR3 shown by SEQ ID NO: 46; (15) LCDR1 shown by SEQ ID NO: 48, LCDR2 shown by SEQ ID NO: 37, LCDR3 shown by SEQ ID NO: 49; (16) LCDR1 shown by SEQ ID NO: 50, LCDR2 shown by SEQ ID NO: 40, LCDR3 shown by SEQ ID NO: 51; (17) LCDR1 shown by SEQ ID NO: 50, LCDR2 shown by SEQ ID NO: 40, LCDR3 shown by SEQ ID NO: 52; (18) LCDR1 shown by SEQ ID NO: 50, LCDR2 shown by SEQ ID NO: 40, LCDR3 shown by SEQ ID NO: 53; (19) LCDR1 shown by SEQ ID NO: 54, LCDR2 shown by SEQ ID NO: 42, LCDR3 shown by SEQ ID NO: 55; (20) LCDR1 shown by SEQ ID NO: 56, LCDR2 shown by SEQ ID NO: 44, LCDR3 shown by SEQ ID NO: 55; and (21) LCDR1, LCDR2, LCDR3 as shown in (1)-(20), but at least one of which includes one, two, three, four, or five amino acids addition, deletion, conservative amino acid substitution, or the combinations thereof.

In one embodiment, the anti-AREG antibody or fragment thereof according to the present invention comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, HCDR3, LCDR1, LCDR2, and LCDR3 are selected from the group consisting of: (1) HCDR1 shown by SEQ ID NO: 1, HCDR2 shown by SEQ ID NO: 2, HCDR3 shown by SEQ ID NO: 3, LCDR1 shown by SEQ ID NO: 21, LCDR2 shown by SEQ ID NO: 22, LCDR3 shown by SEQ ID NO: 23; (2) HCDR1 shown by SEQ ID NO: 1, HCDR2 shown by SEQ ID NO: 2, HCDR3 shown by SEQ ID NO: 4, LCDR1 shown by SEQ ID NO: 21, LCDR2 shown by SEQ ID NO: 22, LCDR3 shown by SEQ ID NO: 24; (3) HCDR1 shown by SEQ ID NO: 5, HCDR2 shown by SEQ ID NO: 2, HCDR3 shown by SEQ ID NO: 6, LCDR1 shown by SEQ ID NO: 25, LCDR2 shown by SEQ ID NO: 26, LCDR3 shown by SEQ ID NO: 27; (4) HCDR1 shown by SEQ ID NO: 7, HCDR2 shown by SEQ ID NO: 8, HCDR3 shown by SEQ ID NO: 9, LCDR1 shown by SEQ ID NO: 28, LCDR2 shown by SEQ ID NO: 29, LCDR3 shown by SEQ ID NO: 30; (5) HCDR1 shown by SEQ ID NO: 7, HCDR2 shown by SEQ ID NO: 10, HCDR3 shown by SEQ ID NO: 9, LCDR1 shown by SEQ ID NO: 31, LCDR2 shown by SEQ ID NO: 32, LCDR3 shown by SEQ ID NO: 30; (6) HCDR1 shown by SEQ ID NO: 7, HCDR2 shown by SEQ ID NO: 8, HCDR3 shown by SEQ ID NO: 11, LCDR1 shown by SEQ ID NO: 33, LCDR2 shown by SEQ ID NO: 34, LCDR3 shown by SEQ ID NO: 30; (7) HCDR1 shown by SEQ ID NO: 7, HCDR2 shown by SEQ ID NO: 8, HCDR3 shown by SEQ ID NO: 12, LCDR1 shown by SEQ ID NO: 35, LCDR2 shown by SEQ ID NO: 34, LCDR3 shown by SEQ ID NO: 30; (8) HCDR1 shown by SEQ ID NO: 1, HCDR2 shown by SEQ ID NO: 13, HCDR3 shown by SEQ ID NO: 14, LCDR1 shown by SEQ ID NO: 36, LCDR2 shown by SEQ ID NO: 37, LCDR3 shown by SEQ ID NO: 38; (9) HCDR1 shown by SEQ ID NO: 1, HCDR2 shown by SEQ ID NO: 13, HCDR3 shown by SEQ ID NO: 140, LCDR1 shown by SEQ ID NO: 39, LCDR2 shown by SEQ ID NO: 40, LCDR3 shown by SEQ ID NO: 38; (10) HCDR1 shown by SEQ ID NO: 1, HCDR2 shown by SEQ ID NO: 13, HCDR3 shown by SEQ ID NO: 140, LCDR1 shown by SEQ ID NO: 41, LCDR2 shown by SEQ ID NO: 42, LCDR3 shown by SEQ ID NO: 38; (11) HCDR1 shown by SEQ ID NO: 1, HCDR2 shown by SEQ ID NO: 13, HCDR3 shown by SEQ ID NO: 140, LCDR1 shown by SEQ ID NO: 43, LCDR2 shown by SEQ ID NO: 44, LCDR3 shown by SEQ ID NO: 38; (12) HCDR1 shown by SEQ ID NO: 1, HCDR2 shown by SEQ ID NO: 15, HCDR3 shown by SEQ ID NO: 16, LCDR1 shown by SEQ ID NO: 39, LCDR2 shown by SEQ ID NO: 40, LCDR3 shown by SEQ ID NO: 38; (13) HCDR1 shown by SEQ ID NO: 1, HCDR2 shown by SEQ ID NO: 15, HCDR3 shown by SEQ ID NO: 16, LCDR1 shown by SEQ ID NO: 45, LCDR2 shown by SEQ ID NO: 42, LCDR3 shown by SEQ ID NO: 46; (14) HCDR1 shown by SEQ ID NO: 1, HCDR2 shown by SEQ ID NO: 15, HCDR3 shown by SEQ ID NO: 16, LCDR1 shown by SEQ ID NO: 47, LCDR2 shown by SEQ ID NO: 44, LCDR3 SHOWN BY SEQ ID NO: 46; (15) HCDR1 shown by SEQ ID NO: 17, HCDR2 shown by SEQ ID NO: 18, HCDR3 shown by SEQ ID NO: 19, LCDR1 shown by SEQ ID NO: 48, LCDR2 shown by SEQ ID NO: 37, LCDR3 shown by SEQ ID NO: 49; (16) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 51; (17) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 52; (18) HCDR1 shown by SEQ ID NO: 17, HCDR2 shown by SEQ ID NO: 18, HCDR3 shown by SEQ ID NO: 20, LCDR1 shown by SEQ ID NO: 50, LCDR2 shown by SEQ ID NO: 40, LCDR3 shown by SEQ ID NO: 53; (19) HCDR1 shown by SEQ ID NO: 17, HCDR2 shown by SEQ ID NO: 18, HCDR3 shown by SEQ ID NO: 20, LCDR1 shown by SEQ ID NO: 54, LCDR2 shown by SEQ ID NO: 42, LCDR3 shown by SEQ ID NO: 55; (20) HCDR1 shown by SEQ ID NO: 17, HCDR2 shown by SEQ ID NO: 18, HCDR3 shown by SEQ ID NO: 20, LCDR1 shown by SEQ ID NO: 56, LCDR2 shown by SEQ ID NO: 44, LCDR3 shown by SEQ ID NO: 55; and (21) HCDR1, HCDR2, HCDR3, LCDR1, LCDR2, LCDR3 as shown in (1)-(20), but at least one of which includes one, two, three, four or five amino acids addition, deletion, conservative amino acid substitution or the combinations thereof.

In some embodiments, the anti-AREG antibody or fragment thereof according to the present invention comprises a heavy chain variable region, and a light chain variable region, wherein the heavy chain variable region has the amino acid sequence selected from the group consisting of SEQ ID NOs: 57-69, and an amino acid sequence having at least 95% sequence identity to any one of SEQ ID NOs: 57-69, and retaining epitope-binding activity, and wherein the light chain variable region has the amino acid sequence selected from the group consisting of SEQ ID NOs: 70-89, and an amino acid sequence having at least 95% sequence identity to any one of SEQ ID NOs: 70-89, and retaining epitope-binding activity.

In some embodiments, the anti-AREG antibody or fragment thereof according to the present invention comprises a heavy chain variable region, and a light chain variable region, wherein the heavy chain variable region and the light chain variable region have the amino acid sequences selected from the group consisting of (1) SEQ ID NO: 57 and SEQ ID NO: 70; (2) SEQ ID NO: 58 and SEQ ID NO: 71; (3) SEQ ID NO: 59 and SEQ ID NO: 72; (4) SEQ ID NO: 60 and SEQ ID NO: 73; (5) SEQ ID NO: 61 and SEQ ID NO: 74; (6) SEQ ID NO: 62 and SEQ ID NO: 75; (7) SEQ ID NO: 63 and SEQ ID NO: 76; (8) SEQ ID NO: 64 and SEQ ID NO: 77; (9) SEQ ID NO: 65 and SEQ ID NO: 78; (10) SEQ ID NO: 66 and SEQ ID NO: 79; (11) SEQ ID NO: 66 and SEQ ID NO: 80; (12) SEQ ID NO: 66 and SEQ ID NO: 81; (13) SEQ ID NO: 67 and SEQ ID NO: 79; (14) SEQ ID NO: 67 and SEQ ID NO: 82; (15) SEQ ID NO: 67 and SEQ ID NO: 83; (16) SEQ ID NO: 68 and SEQ ID NO: 84; (17) SEQ ID NO: 69 and SEQ ID NO: 85; (18) SEQ ID NO: 69 and SEQ ID NO: 86; (19) SEQ ID NO: 69 and SEQ ID NO: 87; (20) SEQ ID NO: 69 and SEQ ID NO: 88; (21) SEQ ID NO: 69 and SEQ ID NO: 89; and (22) two amino acid sequences having at least 95% sequence identity to any one of (1)-(21) respectively, and retaining epitope-binding activity.

In some embodiments, the anti-AREG antibody or fragment thereof according to the present invention is an isotype of IgG, IgM, IgA, IgE, IgD, or the variant thereof. In some embodiments, the anti-AREG antibody or fragment thereof according to the present invention is an isotype of IgG1, IgG2, IgG3, IgG4, or the variant thereof.

In some embodiments, the antibody of the present invention is human monoclonal antibody (mAb), murine mAb, humanized mAb, or chimeric mAb.

Preferably, the humanized monoclonal antibody (mAb) of the present invention comprises constant region derived from human constant region.

Preferably, the humanized monoclonal antibody (mAb) of the present invention has the human light chain constant region derived from kappa or lambda light chain constant region.

Preferably, the humanized monoclonal antibody (mAb) of the present invention has the human heavy chain constant region derived from a human IgG1, IgG2, IgG3, or IgG4 heavy chain constant region.

In some embodiments, the second domain is the ectodomain of TRII or its variant.

In some embodiments, the variant of the ectodomain of TRII is a variant involving a site mutation, and/or a deletion.

In some embodiments, the ectodomain of TRII has the amino acid sequence shown in SEQ ID NO: 90, numbering 1-136 from N-terminus to C-terminus, or an amino acid sequence that is at least 85% identity to SEQ ID NO: 90.

In some embodiments, the site mutation occurs in one or more site mutations selected from K7, T16, D17, R34, R66, K67, K103, and K104 on the basis of the numbering of SEQ ID NO: 90 from N-terminus to C-terminus.

In some embodiments, the site mutation includes one or more site mutations selected from K7Q, T16S, D17N, R34S, R34H, R66S, K67S, K103S, and K104S on the basis of the numbering of SEQ ID NO: 90 from N-terminus to C-terminus.

In some embodiments, the site mutation includes T16S and D17N.

In some embodiments, the site mutation includes K7Q and D17N.

In some embodiments, the site mutation includes K7Q.

In some embodiments, the site mutation includes R34S.

In some embodiments, the site mutation includes R34H.

In some embodiments, the site mutation includes R66S and K67S.

In some embodiments, the site mutation includes K103S and K104S.

In some embodiments, the site mutation includes K7Q and R34S.

In some embodiments, the site mutation includes K7Q, R66S, and K67S.

In some embodiments, the site mutation includes K7Q, K103S, and K104S.

In some embodiments, the site mutation includes K7Q, R34S, R66S, and K67S.

In some embodiments, the site mutation includes K7Q, R34S, K103S, and K104S.

In some embodiments, the site mutation includes K7Q, R66S, K67S, K103S, K104S.

In some embodiments, the site mutation includes K7Q, R34S, R66S, K67S, K103S, and K104S.

In some embodiments, the variant of the ectodomain of TRII carrying one or two mutations selected from K7Q, R34S, R66S, K67S, K103S, and K104S, exhibits decreased clipping of the bi-functional fusion protein.

In some embodiments, the variant of the ectodomain of TRII carrying mutations selected from R34S, R66S, K67S, K103S, and K104S exhibits decreased clipping.

In some embodiments, the variant of the ectodomain of TRII carrying the mutation K7Q exhibits significantly decreased clipping.

In some embodiments, the variant of the ectodomain of TRII is the variant having an N-terminus deletion, preferably, a deletion of four amino acids, seven amino acids, nine amino acids, thirteen amino acids, seventeen amino acids, and twenty-one amino acids on the basis of the numbering of SEQ ID NO: 90 from N-terminus to C-terminus.

In some embodiments, the variant of the ectodomain of TRII is the variant involving the site mutations T16S and D17N, and having an N-terminus deletion of seven amino acids.

In some embodiments, the second domain is the ectodomain of TRII or its variant having an amino acid sequence shown by any one of SEQ ID NOs: 90-107, or an amino acid sequence that is at least 85% identity to any one of SEQ ID NOs: 90-107.

In some embodiments, the C-terminus of the first domain is connected with N-terminus of the second domain, or vice versa, via a linker.

The linker may be a small molecule, a PEG polymer, or a linker peptide. Preferably, the linker is a linker peptide.

In some embodiments, the first domain and the second domain are connected with a short linker peptide of 2 to about 30 amino acids, preferably, 6-26 amino acids. The linker can be rich in glycine for flexibility, as well as serine, threonine, glutamic acid, alanine, or lysine for solubility, and can either connect the C-terminus of the heavy chain or light chain of the anti-AREG antibody with the N-terminus of the ectodomain of TRII or its variant, or vice versa. The linker peptide may be selected from (G₄S)ₙ, (G₄S)ₙG, S(G₄S)ₙG, SG(EAAAK)ₙSG, S(GEGES)ₙG, (EAAAK)ₙ, wherein n is an integer of 1 to 5. In some embodiments, the linker may comprise an amino acid sequence selected from a group consisting of SEQ ID NOs: 108-117. In some embodiments, the C-terminus of the heavy chain or the light chain, preferably, the heavy chain of the anti-AREG antibody is connected with N-terminus of the ectodomain of TRII or its variant directly, or via a linker peptide, versa vice.

In some embodiments, the N-terminus of the heavy chain or the light chain, preferably, the heavy chain of the anti-AREG antibody is connected with C-terminus of the ectodomain of TRII or its variant directly, or via a linker peptide, versa vice.

This bi-functional fusion protein retains the specificity of the original immunoglobulin, despite the introduction of the linker.

In some embodiments, the bi-functional fusion protein according to the present invention comprises the heavy chain or the light chain, preferably, the heavy chain of the anti-AREG antibody connected with the ectodomain of TRII or its variant directly, or via a linker.

In some embodiments, the bi-functional fusion protein according to the present invention comprises the heavy chain or the light chain, preferably, the heavy chain of the anti-AREG antibody, at its N-terminus, connected with C-terminus of the ectodomain of TRII or its variant directly, or via a linker.

In some embodiments, the bi-functional fusion protein according to the present invention comprises the heavy chain or the light chain, preferably, the heavy chain of the anti-AREG antibody, at its C-terminus, connected with N-terminus of the ectodomain of TRII or its variant directly, or via a linker.

In some embodiments, the bi-functional fusion protein according to the present invention comprises the heavy chain of the anti-AREG antibody, at its C-terminus, connected with N-terminus of the ectodomain of TRII via a linker. In some embodiments, the bi-functional fusion protein according to the present invention comprises an amino acid sequence shown in any one of SEQ ID NOs: 118-139, or an amino acid sequence that is at least 85% identity to any one of SEQ ID NOs: 118-139.

Preferably, the bi-functional fusion protein according to the present invention further comprises the light chain of the anti-AREG antibody.

The bi-functional fusion protein according to the present is in the form of heterotetramer.

In some embodiments, the Fc region of the anti-AREG antibody can include a Hinge portion, a CH3 portion and a CH2 portion.

In some embodiments, the Fc region can further include domains that promote heterodimerization, preferably, heterodimerization of the two heavy chains.

In some embodiments, the constant region includes various modifications so as to extend the half-life, improve stability, increase or attenuate ADCC and/or CDC.

The bi-functional fusion protein according to the present invention harbors both anti-TGFβ and anti-AREG activities, possesses the capacity of inhibiting TGFβ and AREG concurrently, and is capable of blocking the pathways relating to TGFβ and AREG simultaneously, and is capable of more efficiently alleviating and treating the pathologies underlying the diseases, including fibrosis, cancers and diseases associated with chronic inflammation. In particular, the bi-functional fusion protein according to the present invention harbors at least a part of the ectodomain of TRII capable of binding to TGFβ ligands, and an antibody or antigen-binding fragment that binds to and neutralizes AREG. The bifunctional protein can block TGFβ and AREG signaling simultaneously. Thus, the bi-functional fusion protein according to the present invention can be used to treat fibrotic diseases, including but not limited to renal fibrosis, hepatic fibrosis, pulmonary fibrosis, in particular, IPF, cancers, and diseases associated with chronic inflammation.

In the second aspect, the present invention provides an isolated nucleic acid, which encodes the bi-functional fusion protein in the first aspect.

In the third aspect, the present invention provides an expression vector, which comprises the isolated nucleic acid in the second aspect.

In the fourth aspect, the present invention provides a host cell, which comprises the isolated nucleic acid in the second aspect, or the expression vector in the third aspect.

The host cell is a conventional host cell in the art, as long as the expression vector of the third aspect can stably express the carried nucleic acid as the bi-functional fusion protein in the first aspect. Preferably, the host cell is a prokaryotic cell and/or a eukaryotic cell, the prokaryotic cell is preferably an E. coli cell such as TG1, BL21, and the eukaryotic cell is preferably HEK293 cell, CHO cells or derived cell lines. The host cell of the present invention can be obtained by transfected with the expression vector of the third aspect. The transfection method is a conventional transfection method in the field, preferably a chemical transfection method, a heat shock method or an electroporation method.

In the fifth aspect, the present invention provides a method for preparing the bi-functional fusion protein in the first aspect.

In some embodiments, the method includes the step of culturing the host cell of the fourth aspect.

In the sixth aspect, the present invention provides a pharmaceutical composition comprising the bi-functional fusion protein in the first aspect and a pharmaceutically acceptable carrier.

In some embodiments, the pharmaceutical composition further comprises other ingredient(s) as active ingredient(s), such as other small molecule drug(s) or antibody(ies) or polypeptide(s) as active ingredient(s).

The pharmaceutical composition is administrated via parenteral, injection, or oral administration. The pharmaceutical composition is in a form suitable for administration, such as a solid, semi-solid, or liquid form, for example, in a form of aqueous solution, non-aqueous solution or suspension, powder, tablet, capsule, granule, injection, or infusion. The pharmaceutical composition is administrated via intravascular, subcutaneous, intraperitoneal, intramuscular, inhalation, intranasal, airway instillation, or intrathoracic instillation. The pharmaceutical composition is administered in the form of an aerosol or spray, for example, nasal administration, or intrathecal, intramedullary, or intraventricular administration, and can also be administered via transdermal, topical, intestinal, or intravaginal, sublingual or rectal administration.

In some embodiments, the bi-functional fusion protein and other active ingredient(s) are administered simultaneously or sequentially.

In the seventh aspect, the present invention provides the use of the bi-functional fusion protein in the first aspect, the isolated nucleic acid in the second aspect, and the pharmaceutical composition in the sixth aspect for the prevention, treatment and/or diagnosis of fibrotic diseases, cancers, and diseases associated with chronic inflammation in a subject. The fibrotic diseases include but not limited to renal fibrosis, hepatic fibrosis, and pulmonary fibrosis, in particular, IPF.

In a ninth aspect, the present invention provides a method for preventing, treating, and/or diagnosing fibrotic diseases, cancers, and diseases associated with chronic inflammation in a subject, which comprises administering to the subject a therapeutically effective amount of the bi-functional fusion protein in the first aspect. The fibrotic diseases include but not limited to renal fibrosis, hepatic fibrosis, and pulmonary fibrosis, in particular, IPF.

The bi-functional fusion protein of the present invention has the following technical effects:
1. as an antagonist of TGFβ and AREG;
2. as a single bifunctional protein that possesses the capacity of inhibiting TGFβ and AREG, can efficiently block the two drivers for fibrogenesis and elicit a synergistic effect to treat tissue fibrosis, in particular pulmonary fibrosis;
3. as a single bifunctional protein that blocks TGFβ and AREG signaling simultaneously;
4. as a single bifunctional protein that specifically binds to TGFβ and AREG;
5. having the capacity to inhibit pEGFR induced by AREG;
6. having capacity to bind to the TGFβ ligands and therefore inhibit downstream activation of the TGFβ signaling pathway;
7. the bi-functional fusion protein involving the variant of the ectodomain of TRII carrying one or two mutations selected from K7Q, R34S, R66S, K67S, K103S, and K104S, exhibits decreased clipping of the bi-functional fusion protein;
8. the bi-functional fusion protein involving the variant of the ectodomain of TRII carrying mutations selected from R34S, R66S, K67S, K103S, and K104S exhibits decreased clipping;
9. the bi-functional fusion protein involving the variant of the ectodomain of TRII carrying the mutation K7Q exhibits significantly decreased clipping;
10. the bi-functional fusion protein involving the variant of the ectodomain of TRII having a N-terminus deletion of four amino acids, seven amino acids, nine amino acids, thirteen amino acids, seventeen amino acids, or twenty-one amino acids on the basis of the numbering of SEQ ID NO: 90 from N-terminus to C-terminus shows better stability and less clipping.

### Definitions

Transforming growth factor beta (TGFβ) is a pleiotropic cytokine that plays an essential role under physiological and pathological conditions. TGFβ isoforms, namely β1, β2, and β3, are homodimeric polypeptides around 25 kDa. These ligands signal through cell surface receptors, including transforming growth factor R receptor I, II, and III (TRI, TRII, TRIII), and intracellular SMAD effector proteins such as SMAD 2 and 3. The transduction of the signal affects a series of cellular processes including cell survival, proliferation, differentiation, cell motility, and extracellular matrix (ECM) production.

Amphiregulin, AREG, is a low-affinity ligand in the epidermal growth factor (EGF) family. AREG protein is synthesized from a 252 amino acid transmembrane precursor, which is subjected to proteolytic cleavage within its ectodomain by cell membrane proteases, mainly TACE (tumor necrosis factor-α-converting enzyme). Mature soluble AREG then activates downstream signaling by binding directly to epidermal growth factor receptor (EGFR). This can elicit major intracellular signaling cascades including MAPK/ERK signaling to govern cell survival, proliferation, and motility.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The products and methods disclosed herein encompass polypeptides and polynucleotides having the sequences specified, or sequences identical or similar thereto, e.g., sequences having at least about 85% or 95% sequence identity (identical) to the sequence specified. In the context of an amino acid sequence, the term “85% or 95% sequence identity (identical)” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 86%, 87%, 88%, 89%, 90%, 91%, 92%, 93%, 94%, 95%, 96%, 97%, 98% or 99% identity to a reference sequence, e.g., a sequence provided herein.

In the context of nucleic acid, the term “85% or 95% sequence identity (identical)” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 86%, 87%, 88%, 89%, 90%, 91%, 92%, 93%, 94%, 95%, 96%, 97%, 98% or 99% identity to a reference sequence, e.g., a sequence provided herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The terms “polypeptide”, “peptide” and “protein” are used interchangeably herein to refer to polymers of amino acids of any length.

The terms “nucleic acid”, “nucleic acid sequence”, “nucleotide sequence”, or “polynucleotide sequence”, and “polynucleotide” are used interchangeably.

As used herein, the term “antibody” refers to a protein, e.g., an immunoglobulin chain or fragment thereof, comprising at least one immunoglobulin variable domain sequence. The term “antibody” includes, for example, a monoclonal antibody (including a full length antibody which has an immunoglobulin Fc region). In an embodiment, an antibody comprises a full length antibody, or a full length immunoglobulin chain. In an embodiment, an antibody comprises an antigen binding or functional fragment of a full length antibody, or a full length immunoglobulin chain. As used herein, an antibody “binds to” an antigen as such binding is understood by one skilled in the art. In one embodiment, an antibody binds to an antigen with a dissociation constant (KD) of about 1×10⁻⁵M or less, 1×10⁻⁶M or less, or 1×10⁻⁷M or less.

For example, an antibody can include a heavy (H) chain variable domain sequence (abbreviated herein as VH), and a light (L) chain variable domain sequence (abbreviated herein as VL). In an embodiment, an antibody comprises or consists of a heavy chain and a light chain. In another example, an antibody includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibodies can be monoclonal or polyclonal. An antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

The terms “antibody fragment” or “antigen-binding fragment”, as used herein, is a portion of an antibody such as F(ab′)₂, F(ab)₂, Fab′, Fab, Fv, scFv and the like. An antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Examples of antigen-binding fragments of an antibody include: (i) a Fab fragment, a monovalent fragment consisting of the VL, VH, CK and CH portions; (ii) a F(ab′)₂ fragment, a bivalent fragment comprising two Fab fragments linked by a disulfide bridge at the hinge region; (iii) a Fd fragment consisting of the VH and CH portions; (iv) a Fv fragment consisting of the VL and VH portions of a single arm of an antibody, (v) a diabody (dAb) fragment, which consists of a VH portion; (vi) a camelid or camelized variable portion; (vii) a single chain Fv(scFv); (viii) a single portion antibody. These antibody fragments may be obtained using any suitable method, including conventional techniques known to those with skill in the art, and the fragments can be screened for utility in the same manner as are intact antibodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (VH) and light chains (VL) of immunoglobulins.

In some aspects, the regions are connected with a short linker peptide of 2 to about 30 amino acids, preferably, 6-26 amino acids. The linker can be rich in glycine for flexibility, as well as serine, threonine, glutamic acid, alanine, or lysine, and can either connect the N-terminus of the VH of the anti-AREG antibody or fragment thereof with the C-terminus of the ectodomain of TRII or its variant, or vice versa.

The light and heavy chains are divided into regions of “constant” and “variable”. The variable domains of both the light (VL) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. The N-terminal portion is a variable region and at the C-terminal portion is a constant region; the CH3 and CK portions actually comprise the carboxy-terminus of the heavy and light chain, respectively.

The variable region allows the antibody to selectively recognize and specifically bind epitopes on antigens. The VL portion and VH portion, or subset of the complementarity determining regions (CDRs), of an antibody combine to form the variable region that defines a three dimensional antigen-binding site. This quaternary antibody structure forms the antigen-binding site present at the end of each arm of Y. More specifically, the antigen-binding site is defined by three CDRs on each of the VH and VK chains (i.e. HCDR1, HCDR2, HCDR3, LCDR1, LCDR2 and LCDR3).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR is determined using the well-known scheme described by Kabat et al. (1991), “Sequences of Proteins of Immunological Interest,” 5th Ed. Public Health Service, National Institutes of Health, Bethesda, MD (“Kabat” numbering scheme).

Each VH and VL typically includes three CDRs and four FRs, arranged from amino-terminus to carboxy-terminus in the following order: FR1, CDR1, FR2, CDR2, FR3, CDR3, FR4.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

As used herein, the term “epitope” refers to the moieties of an antigen (e.g., human AREG) that specifically interact with an antibody. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

The terms “monoclonal antibody” or “monoclonal antibody composition” as used herein refer to a preparation of antibodies of single molecular composition. A monoclonal antibody composition displays a single binding specificity and affinity for a particular epitope. A monoclonal antibody can be made by hybridoma technology or by methods that do not use hybridoma technology (e.g., library selection, and screening, or recombinant methods).

The antibody can be a polyclonal or a monoclonal antibody. In other embodiments, the antibody can be recombinantly produced, e.g., produced by yeast display, phage display, or by combinatorial methods.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a murine (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen or its fragment of interest are used to produce hybridomas that secrete human mAbs with specific affinities for epitopes.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Also within the scope of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In yet other embodiments, the antibody has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4.

It is understood that the molecules of the invention may have additional conservative or nonessential amino acid substitutions, which do not have a substantial effect on their functions.

A “conservative amino acid substitution” is one in which the amino acid residue is replaced with an amino acid residue having a similar side chain, as shown in Table 1. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

TABLE 1
Conservative amino acid substitutions:

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4-Diamino-butyric acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

### BRIEF DESCRIPTION OF THE DRAWINGS

FIG. 1 shows structure of an exemplary anti-AREG/TRII bi-functional fusion protein.

FIG. 2 shows characterization of the clipping species in anti-AREG/TRII variants with different linkers.

FIG. 3 shows the hotspots (bold italic letters) of cleavage in anti-AREG/TRII.

FIG. 4 shows K7 mutation (014) in anti-AREG/TRII decreases clipping.

FIG. 5 shows N-terminus deletion of TRII in anti-AREG/TRII variants decreases heavy chain clipping.

FIG. 6 shows N-terminus deletion of TRII (013, 017, 028, 029, 030) in anti-AREG/TRII decreases heavy chain clipping.

FIG. 7 shows the purity of anti-AREG/TRII variant 013, 029, and 030.

FIG. 8 shows the inhibition of pEGFR by variant 010.

FIG. 9 shows the inhibition of the SRE reporter by variant 010, 013, and 030.

FIG. 10 shows variant 010, 013, and 030 blocks the nuclear localization of pSMAD2.

FIG. 11 shows variant 013 inhibits pSMAD2 in an immunoblotting assay.

FIG. 12 shows the inhibition of TGFβ signaling by variant 010, 013, and 030 in a SBE luciferase reporter assay.

FIG. 13 shows the simultaneous targeting of AREG and TGFβ by one bifunctional anti-AREG/TRII fusion protein.

FIG. 14 shows the pharmacokinetic profile of variant 010, 013, and 030.

FIG. 15 shows the efficacy of anti-AREG/TRII fusion protein in the Cdc42 AT2 null fibrosis model.

### DETAILED DESCRIPTION

### Description of Particular Embodiments of the Invention

The descriptions of particular embodiments and examples are provided by way of illustration and not by way of limitation. Those skilled in the art will readily recognize a variety of noncritical parameters that could be changed or modified to yield essentially similar results.

The following Table 2 schematically shows the structure of different anti-AREG/TRII variants.

TABLE 2

| Variants | |
|---|---|
| No. | Seq (on C-terminal of heavy chain) |
| 001 | -S(G4S)4G-TRII 1-136 |
| 002 | -S(G4S)3G-TRII 1-136 |
| 003 | -S(G4S)2G-TRII 1-136 |
| 004 | -S(G4S)1G-TRII 1-136 |
| 005 | -SG(EAAAK)4SG-TRII 1-136 |
| 006 | -SG(EAAAK)3SG-TRII 1-136 |
| 007 | -SG(EAAAK)2SG-TRII 1-136 |
| 008 | -S(GEGES)4G-TRII 1-136 |
| 009 | -(EAAAK)4-TRII 1-136 |
| 010 | -(G4S)4G-TRII 1-136 |
| 011 | -(G4S)4G-TRII 8-136 T16S/D17N |
| 012 | -(G4S)4G-TRII 1-136 K7Q/D17N |
| 013 * | -(G4S)4G-TRII 22-136 |
| 014 | -(G4S)4G-TRII 1-136 K7Q |
| 015 | -(G4S)4G-TRII 1-136 R34S |
| 016 | -(G4S)4G-TRII 1-136 R34H |
| 017 | -(G4S)4G-TRII 1-136 R66S/K67S |
| 018 | -(G4S)4G-TRII 1-136 K103S/K104S |
| 019 | -(G4S)4G-TRII 1-136 K7Q/R34S |
| 020 | -(G4S)4G-TRII 1-136 K7Q/R66S/K67S |
| 021 | -(G4S)4G-TRII 1-136 K7Q/K103S/K104S |
| 022 | -(G4S)4G-TRII 1-136 K7Q/R34S/R66S/K67S |
| 023 | -(G4S)4G-TRII 1-136 K7Q/R34S/K103S/K104S |
| 024 | -(G4S)4G-TRII 1-136 K7Q/R66S/K67S/K103S/K104S |
| 025 | -(G4S)4G-TRII 1-136 K7Q/R34S/R66S/K67S/K103S/K104S |
| 026 | -(G4S)4G-TRII 8-136 |
| 027 | -(G4S)4G-TRII 5-136 |
| 028 | -(G4S)4G-TRII 10-136 |
| 029 | -(G4S)4G-TRII 14-136 |
| 030* | -(G4S)4G-TRII 18-136 |

### EXAMPLES

### Example 1. Production of Anti-AREG/TRII Bi-Functional Fusion Protein

Anti-AREG/TRII is an anti-AREG antibody-ectodomain of transforming growth factor R receptor II (TGFβRII, TRII) bi-functional protein. The light chain variable region of the molecule is identical to that of the anti-AREG antibody (SEQ ID No.:70-89). The heavy chain of the molecule is a fusion protein comprising the heavy chain of the anti-AREG antibody (SEQ ID No.:57-69) genetically fused to the N-terminus of the soluble TRII (SEQ ID No.:90-107) via a flexible linker (SEQ ID No.:108, 109, 113, and 116-117). At the fusion junction, the C-terminal lysine residue of the antibody heavy chain was mutated to alanine to reduce proteolytic cleavage.

The following exemplary procedure was used to construct the plasmid.

The fragments were amplified by the polymerase chain reaction (PCR) (TOYOBO, KOD-201). The PCR product was separated on a 1.5% agarose gel after electrophoresis and recovered using a DNA purification kit (Magen, D2111-03). The fragments and vectors were separately digested with the restriction enzymes and ligated using a T4 DNA ligase (New England Biolabs, M0202L). The constructs following ligation were transformed into the E. coli Top10 strain (CWBIO, CW0807) for positive clone selection. The cloned plasmids were used for protein expression in a eukaryotic expression system.

The following exemplary procedure was used to produce a protein.

Two expression vectors with the heavy chain and the light chain were transfected into FreeStyle™ 293-F cells (Invitrogen, R79007) at a 1:1 ratio. The day before transfection, 293-F cells were subcultured and expanded to allow overnight growth. On the day of transfection, cells were collected by centrifugation and then resuspended in fresh FreeStyle™ 293 expression medium (Gibco, 12336-018) to a final density of 1.2×10⁶ cells/mL. The plasmids with a final concentration at 1 μg/mL were transiently cotransfected at the indicated molar ratios with polyethylenimine (Polysciences, 23966). Cell culture supernatant was harvested 5-6 days after the transfection.

### Example 2. Characterization of Anti-AREG/TRII Bi-Functional Fusion Proteins with Different Linkers

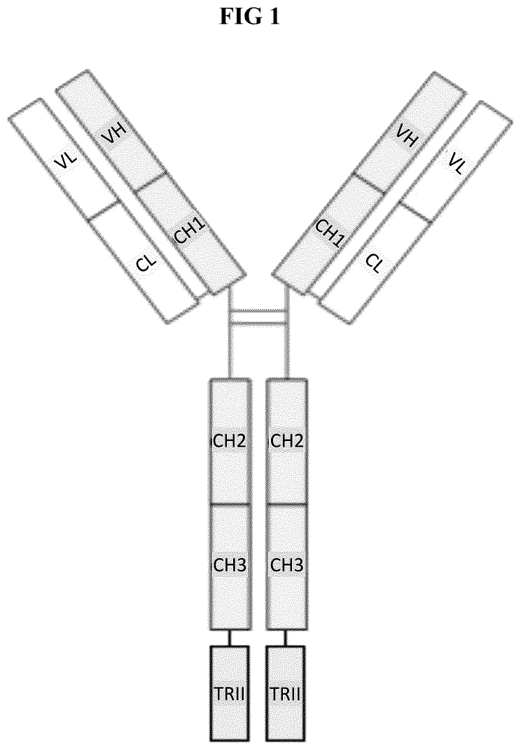

The exemplary anti-AREG/TRII bi-functional fusion protein (anti-AREG/TRII) 001, 005, 008, and 009 contain a light chain (SEQ ID No:70-89), and a heavy chain fused to TRII via different linkers (SEQ ID No.:118-121). The structure of the bifunctional fusion protein is shown in FIG. 1.

The following exemplary procedure was used to evaluate the clipping of the fusion proteins. Fusion proteins were harvested after transfection, and then purified by one-step protein A chromatography. All samples were adjusted to a concentration of 0.5 mg/mL, and then incubated at 37° C. for stability tests. The samples were analyzed by Sodium dodecyl-sulfate polyacrylamide gel electrophoresis (SDS-PAGE) under reducing conditions.

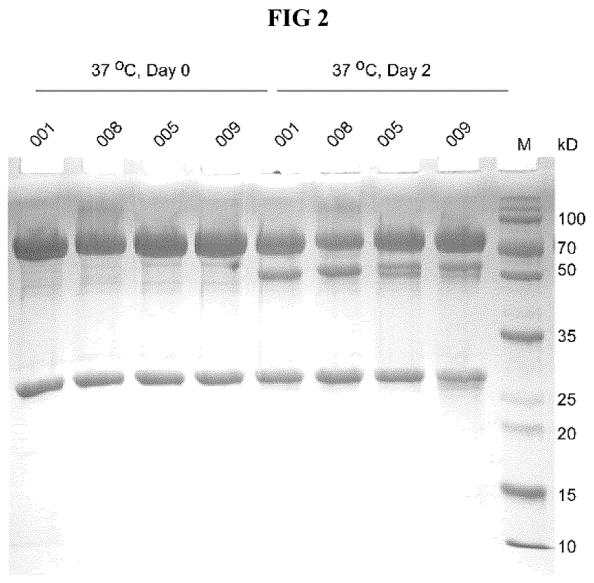

FIG. 2 shows that two bands of approximately 25 kDa and 70 kDa were detected in all samples after SDS-PAGE. These two bands represented the light chain and heavy chain of the bi-functional protein. Additional bands around 50-60 kDa were also detected after 2 days of incubation at 37° C., suggesting that various clipping species were present following additional incubation of the variants.

### Example 3. Determination of Cleavage Sites on Anti-AREG/TRII Bi-Functional Fusion Proteins

Anti-AREG/TRII with different linkers showed varying degrees of clipping, which appeared as molecules with different molecular weight on SDS-PAGE. Therefore, further investigation was performed to identify the clipped sites.

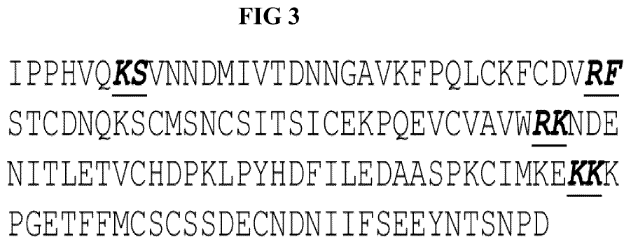

0.5 mg/mL 001 or 009 was incubated at 37° C. After 2 days of incubation, the samples were loaded into a gel. Clipped fragments were separated and recovered. The fragments were sent for mass spectrometry (MS) analysis (LTQ Orbitrap, ThermoFisher Scientific). The analyzed result indicated that Lys7/Ser8, Arg34/Phe35, Arg66/Lys67, and Lys103/Lys104 were hotspots of TGFβ trap clipping, as shown in FIG. 3.

Prediction of potential cleavage sites was performed on https://web.expasy.org/peptide_cutter/. The results from these analyses showed that Lys7/Ser8, Arg34/Phe35, Arg66/Lys67, and Lys103/Lys104 were also potential cleavage sites.

### Example 4. Investigation of Anti-AREG/TRII Variants that Reduce Clipping

In order to develop TRII-containing molecules that exhibit significantly reduced clipping, various traps with TRII mutants (SEQ ID NO: 90-107) were designed.

The exemplary anti-AREG/TRII contains a light chain (SEQ ID NO: 83) and a heavy chain (SEQ ID No.:122-139) that was fused to TRII variants. 010 was an anti-AREG/TRII variant with wild type TRII ectodomain. 014, 015, 016, 017, and 018 were anti-AREG/TRII variants with TRII carrying one or two mutations at the aforementioned sites. The purified samples were incubated at 37° C. for 3 days. The clipping species were evaluated by the method described in Example 3.

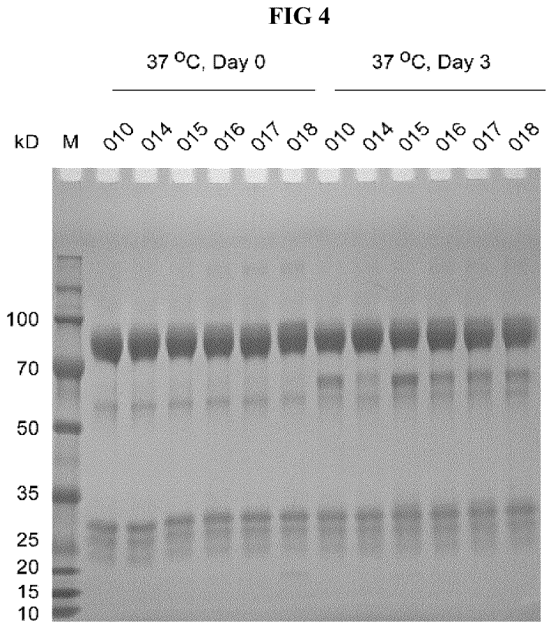

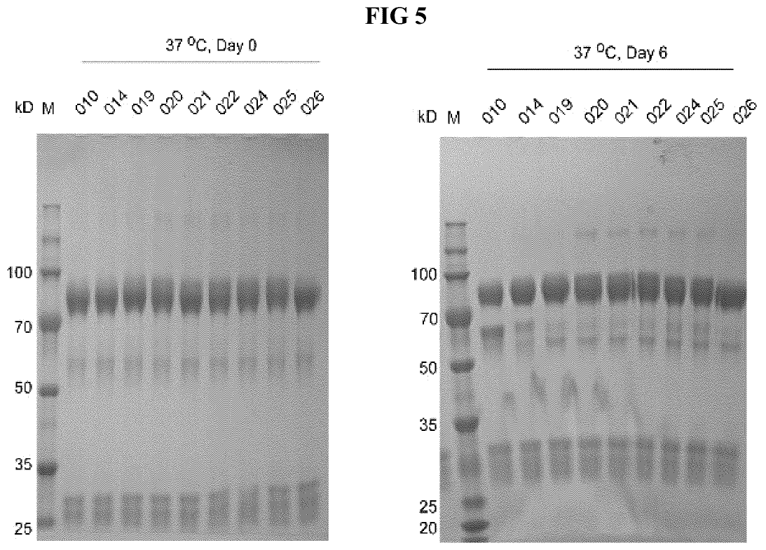

FIG. 4 shows that K7 mutation (014) exhibited significantly decreased heavy chain clipping. R34(016), R66/K67(017), and K103/K104(018) mutations also yielded decreased clippings, compared with 010.

Given that K7 was the most critical residue mediating clipping, a new variant with a deletion of 7 residues in N-terminus of TRII, 026, was designed. A combination of K7 mutation with R34, R66/K67, and K103/K104 mutations (019, 020, 021, 022, 024, 025, and 026) were also designed and evaluated for clipping. As shown in FIG. 5, among all variants, 026 showed the least clipping after incubation at 37° C. for 6 days.

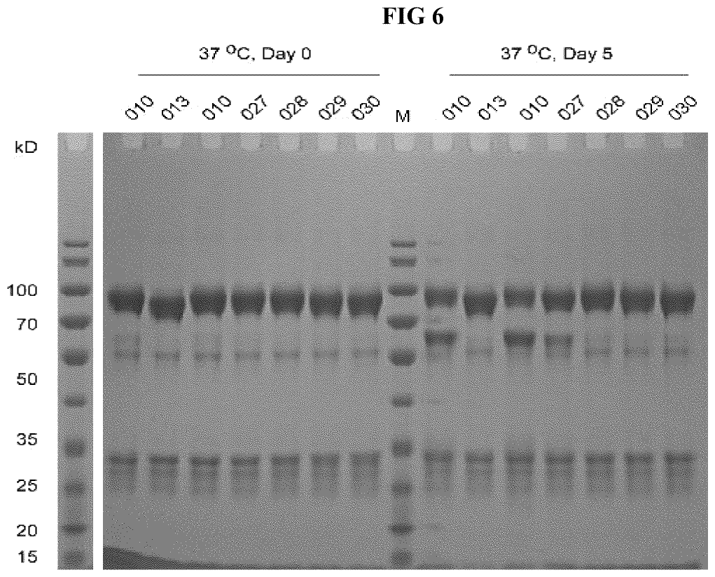

Deletion variant 026 showed better stability, compared with site mutations or combinations of site mutations. Therefore, additional deletion variants were designed, including a 4-residue deletion (027), a 9-residue deletion (028), a 13-residue deletion (029), a 17-residue deletion (030), and a 21-residue deletion (013). After 5 days of incubation at 37° C., 029, 030, and 013 showed less clippings than 010 in SDS-PAGE as shown in FIG. 6.

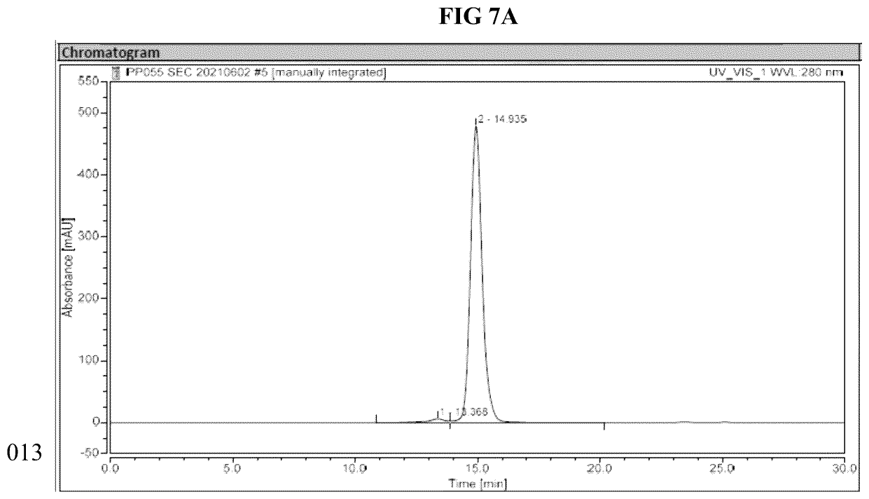

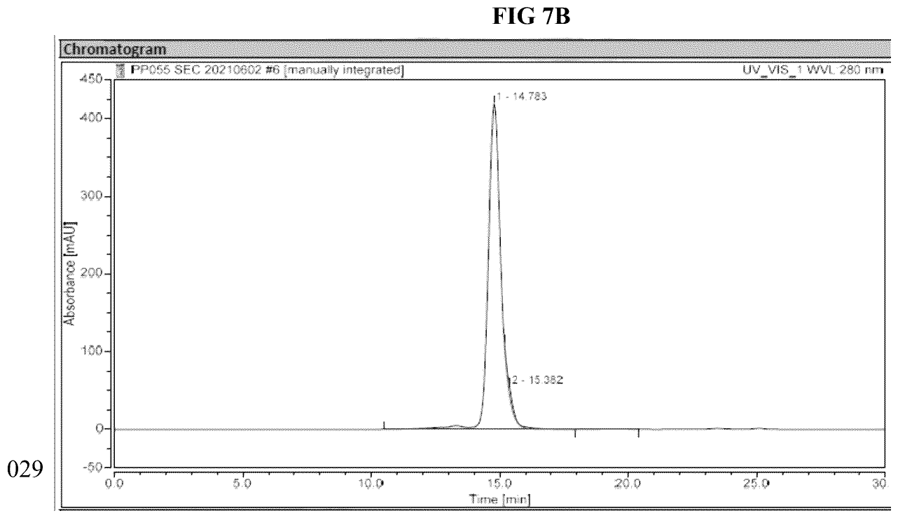

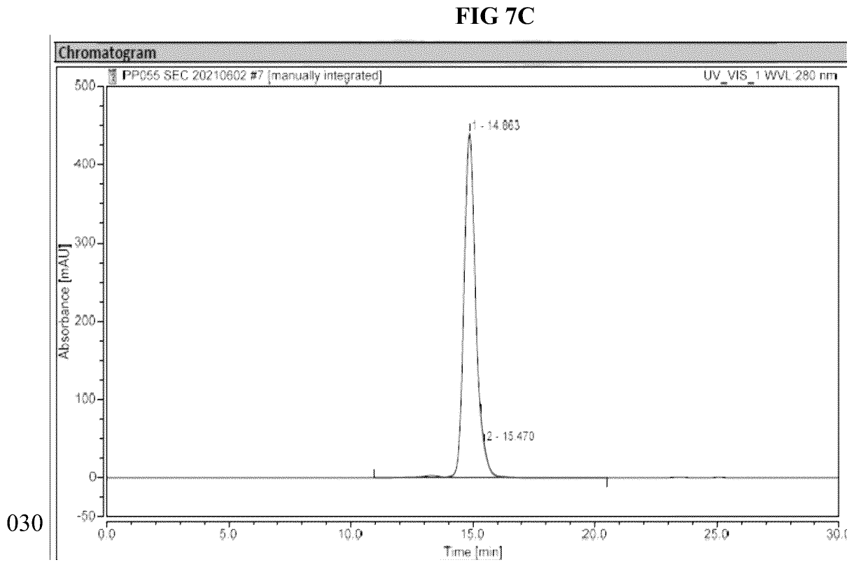

Size exclusion chromatography (SEC) was also applied to evaluate the purity of anti-AREG/TRII variants. The exemplary profile of 013, 029, and 030 were shown in FIG. 7. The purity was over 98% after purification. The level of high molecular weight (HMW) and low molecular weight (LMW) was less than 2%.

### Example 5. Binding of Anti-AREG/TRII Variants to AREG and TGFβ by SPR

Surface plasmon resonance (SPR) assay was deployed to characterize the binding kinetics of anti-AREG/TRII variants to AREG and TGFβs.

The following exemplary procedure was used to measure the equilibrium dissociation constant.

SPR measurement was performed using Biacore T200 instrument (GE Life Sciences). The samples were captured on the surface of Protein A/G CM5 biosensor chip. Human TGFβ3 (R&D, 243-B3-010) were examined for their binding to the different variants. TGFβ proteins in serial dilutions were injected over the anti-AREG/TRII variants-bound surface, and this was followed by a dissociation phase. Association rates (ka) and dissociation rates (kd) were calculated using the one-to-one Langmuir binding model (BIA Evaluation Software, GE Life Sciences). The KD was calculated as the ratio of kd to ka.

As shown in Table 3, 010, 013, and 030 bind to TGFβ 1/2/3 and AREG at similar kinetics.

TABLE 3

| | | ka (1/Ms) | kd (1/s) | KD (M) |
|---|---|---|---|---|
| TGFβ1 | 010 | 4.69E+07 | 3.50E−05 | 7.46E−13 |
| | 013 | 1.12E+08 | 5.71E−05 | 5.52E−13 |
| | 030 | 8.28E+07 | 5.94E−05 | 7.18E−13 |
| TGFβ2 | 010 | 1.91E+07 | 1.24E−02 | 6.50E−10 |
| | 013 | 5.85E+07 | 3.84E−01 | 6.57E−09 |
| | 030 | 1.34E+08 | 9.01E−01 | 6.71E−09 |
| TGFβ3 | 010 | 1.02E+08 | 6.67E−05 | 6.52E−13 |
| | 013 | 7.72E+07 | 1.48E−04 | 1.92E−12 |
| | 030 | 6.13E+07 | 1.28E−04 | 2.08E−12 |
| AREG | 010 | 1.93E+07 | 6.87E−04 | 3.56E−11 |
| | 013 | 1.95E+07 | 7.18E−04 | 3.69E−11 |
| | 030 | 1.70E+07 | 6.14E−04 | 3.59E−11 |

### Example 6. Determination of EGFR Inhibition by Anti-AREG/TRII Variants Via Examining Phosphorylated EGFR

AREG binds to epidermal growth factor receptor (EGFR) and leads to the activation of the receptor, which could be measured by phosphorylated EGFR (pEGFR). The exemplary anti-AREG/TRII variant 010 was examined for its capacity to inhibit pEGFR induced by AREG.

The following exemplary procedure was used. A431 cells were seeded in a 6-well plate at 2×10⁵ cells/well and cultured at 37° C. overnight. Cells were starved with serum free medium for 2 hours the next day and then treated with recombinant human AREG (Peprotech, 96-100-55B-50) at 10 nM and anti-AREG/TRII variants at various concentrations for 1 hour. Treated cells were lysed and samples were subjected to Western Blotting. Primary antibodies including Anti-pEGFR (Abcam, ab40815), anti-EGFR (Cell Signaling technology, 2232) and anti-GAPDH (Nakasugi Jinqiao, TA-08) were used for this assay.

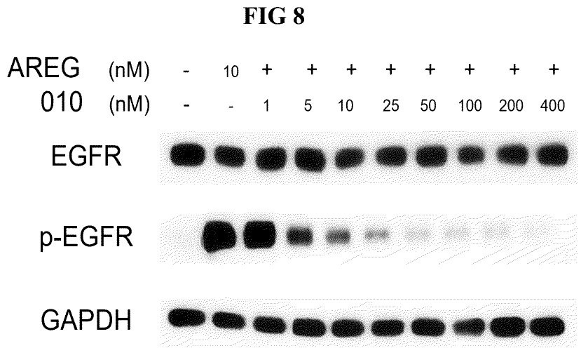

FIG. 8 shows that variant 010 inhibits AREG-induced pEGFR at an IC50 of 5.56 nM. This data suggests that variant 010 can efficiently block AREG-induced activation of EGFR.

### Example 7. Characterization of Anti-AREG/TRII Variants by a MAPK/ERK Signaling Luciferase Reporter

The inhibition of the anti-AREG/TRII variants on AREG-EGFR downstream signaling was further examined using a serum response element (SRE) luciferase reporter. This reporter is commonly used to assess EGFR-MAPK/ERK signaling.

The following exemplary procedure was used. A SRE-luciferase reporter was constructed and transfected to HEK293T cells in a 96-well plate using the Lipo 3000 transfection reagent kit (Invitrogen, L3000-015). Each well was co-transfected with a TK-Renilla plasmid as an internal control. After 6 hours of transfection, cells were serum starved and then treated with the recombinant human AREG (Peprotech, 96-100-55B-50) and anti-AREG/TRII variants for 6 hours. A Dual-Glo® Luciferase Assay System (Promega, E2940) was used to examine the luciferase signal after the treatment.

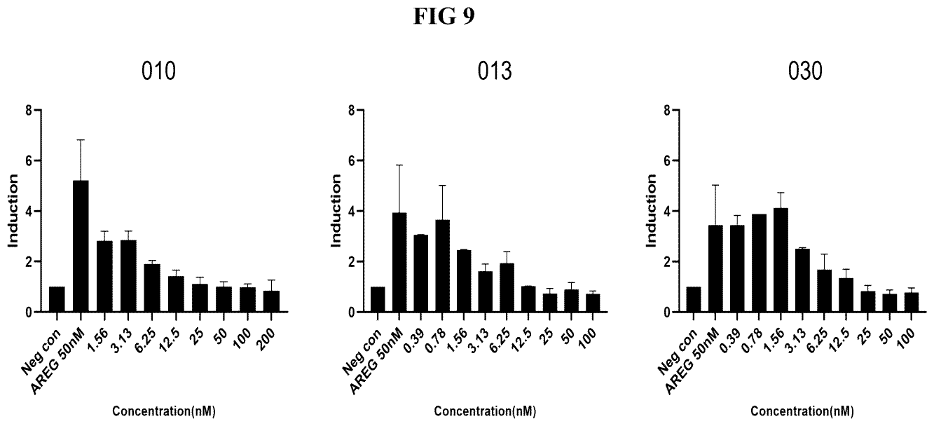

FIG. 9 shows that anti-AREG/TRII variants, including 010, 013, and 030, inhibit the expression of the SRE reporter induced by AREG with an IC99 around 25 nM. This data suggests that anti-AREG/TRII variants can efficiently inhibit EGFR-MAPK/ERK signaling that is included by AREG.

### Example 8. Assessing the Inhibition of Anti-AREG/TRII Variants on TGFβ Signaling by pSMAD2 Nuclear Localization

The ectodomain of transforming growth factor R receptor II in the anti-AREG/TRII fusion protein was designed to sequester the TGFβ ligands and therefore inhibit downstream activation of the TGFβ signaling pathway. Nuclear phosphorylated SMAD2 (pSMAD2) localization is frequently used to assess the activation of this pathway. The effect of anti-AREG/TRII variants on pSMAD2 nuclear localization, namely TGFβ signaling activation, was examined by pSMAD2 immunofluorescence staining.

The following exemplary procedure was used to examine pSMAD2 nuclear localization. A549 cells were seeded at a density of 5,000 cells per well into a 96-well microplate and cultured with DMEM with 10% FBS at 37° C. overnight. On the next day, cells were first serum starved and then treated with 0.078 nM of TGFβ1 (R&D, 240-B-101) and anti-AREG/TRII variants at 0.39 nM and 1.95 nM, respectively, for 6 hours. Cells were rinsed with PBS, fixed, and stained with an anti-pSMAD2 antibody (Cell Signaling technology, 18338) overnight at 4° C. A secondary antibody (Jackson Immuno Research, 711-064-152) was applied the next day before samples were treated with the Elite ABC kit (VECTOR, PK-6100) for 30 min followed by tyramide fluorescein staining (Pekin Elmer, FP1013). The 20× air lens of Opera LX from Pekin Elmer was used to capture the fluorescent images.

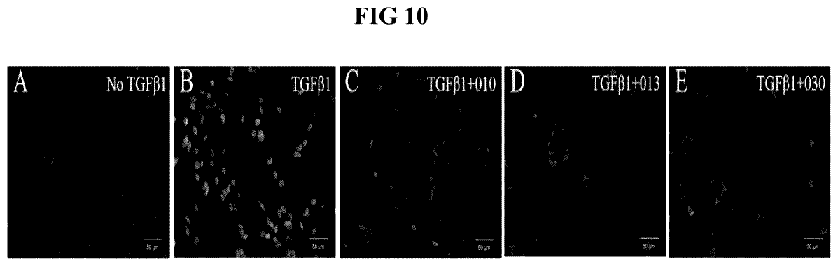

As shown in FIG. 10, anti-AREG/TRII variants 010, 013, and 030 at 1.95 nM efficiently blocked pSMAD2 nuclear localization induced by 1 ng/ml of TGFβ1, suggesting that anti-AREG/TRII variants 010, 013, and 030 are capable of binding to and sequestering TGFβ1 and inhibiting downstream TGFβ signaling.

### Example 9. Quantification of the Inhibition of Anti-AREG/TRII Variants on TGFβ Signaling by pSMAD2 Immunoblotting

The expression level of pSMAD2 protein was also used to quantify the inhibitory effects of anti-AREG/TRII variants on TGFβ1 through Western Blot (WB).

The following exemplary procedure was used. A549 cells were seeded in a 6-well plate at 2×10⁵ cells/well and cultured at 37° C. overnight. Cells were starved with serum free medium for 2 hours on the next day and treated with recombinant human TGFβ1 (R&D, 240-B-101) and anti-AREG/TRII at various concentrations for 2 hours. Treated cells were lysed and samples were subjected to Western Blot analysis. Primary antibodies included anti-pSMAD2 (Cell Signaling technology, 18338), anti-SMAD2/3 (Cell Signaling technology, 8685), and anti-GAPDH (Nakasugi Jinqiao, TA-08).

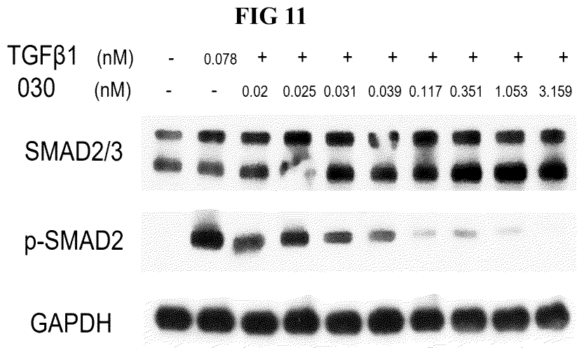

FIG. 11 shows that variant 030 inhibits pSMAD2 with an IC50 at 0.034 nM when 1 ng/mL of TGFβ1 was added to the culture. This data shows that the TRII ECD portion of the anti-AREG/TRII can efficiently trap ligands such as TGFβ1 and inhibit downstream signaling activation.

### Example 10. Quantification of the Inhibition of Anti-AREG/TRII Variants on TGFβ Signaling by a SBE Reporter

The inhibitory effect of the exemplary anti-AREG/TRII variants 010, 013, and 030 on TGFβ downstream signaling was further quantified using a SMAD-binding element (SBE) luciferase reporter. This reporter is frequently used to assess the activity of TGFβ signaling.

The following exemplary procedure was used. A SBE-luciferase reporter was constructed and transfected to HEK293T cells in a 96-well plate using the Lipo 3000 transfection kit (Invitrogen, L3000-015). Each well was co-transfected with a TK-Renilla plasmid as an internal control. After 6 hours of transfection, cells were serum starved and then treated with human TGFβ 1 (R&D, 240-B-101) and anti-AREG/TRII for 6 hours. A Dual-Glo® Luciferase Assay System (Promega, E2940) was then used to examine the luciferase signal after the treatment.

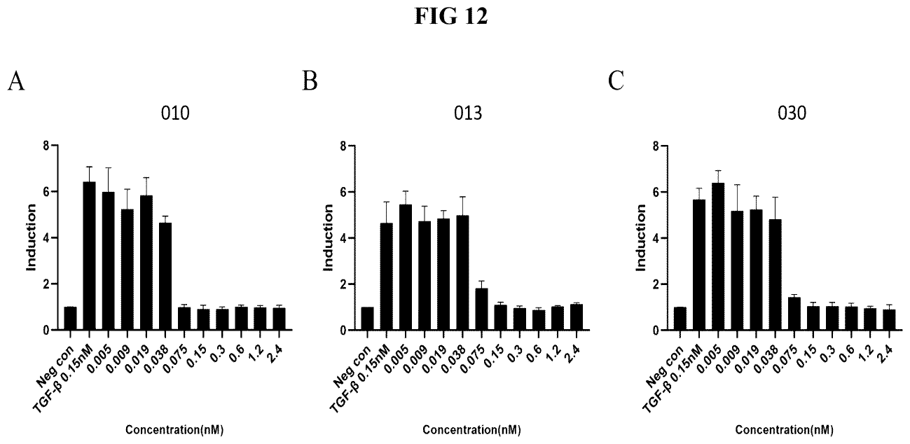

As shown in FIG. 12, anti-AREG/TRII variants 010, 013, and 030 blocked the induction of the SBE reporter by TGFβ1 (0.015 nM) with an IC50 at 0.045, 0.085, and 0.06 nM, respectively.

### Example 11. Simultaneous Targeting of Both AREG and TGFβ by Anti-AREG/TRII Variants

The concept behind anti-AREG/TRII bi-functional fusion protein is to simultaneously target AREG and TGFβ ligands through one molecule. To test this, a CHO-hAREG cell line was generated to overexpress AREG EGF-like domain that is mostly associated with the cell membrane. When anti-AREG/TRII is added to CHO-hAREG cells, the bi-functional fusion protein will bind to membrane AREG EGF-like domain through the anti-AREG portion. The membrane-bound bifunctional molecule can also block the activation of TGFβ signaling through trapping the free TGFβ ligands.

The following exemplary procedure was used. CHO and CHO-hAREG cells were harvested and seeded at a density of 8,000 cells per well into a 96-well microplate in DMEM supplemented with 10% FBS. The plate was incubated at 37° C. in a CO₂ incubator overnight. On the next day, cells were starved for 4 h in the serum-free DMEM and then treated with DMEM containing 10 nM variants for 2 h. After washing off the free variants, 0.078 nM of TGFβ1 was added and incubated with the cells for 1 h in the CO₂ incubator. After the treatment, cells were rinsed twice with PBS, fixed with 4% PFA, and then stained with an anti-pSMAD2 antibody (Cell Signaling technology, 18338). After staining, the 20× air lens of Pekin Elmer's high-content cell analysis system was used to capture the fluorescence images.

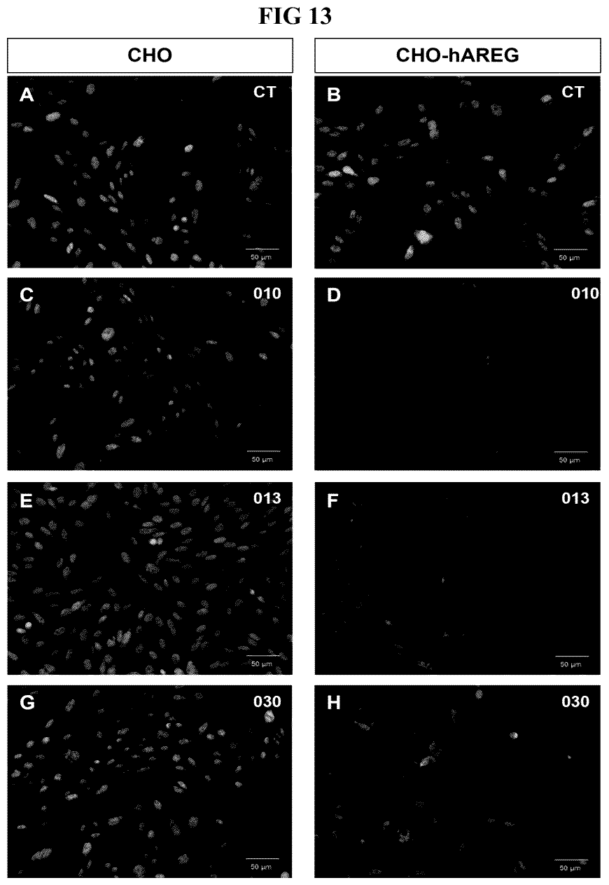

As shown in FIG. 13, variant 010, 013, and 030 can efficiently block TGFβ1-induced signaling while binding to AREG, demonstrating that the bifunctional fusion molecule can simultaneously block AREG and TGFβ signaling.

### Example 12. Pharmacokinetics of Anti-AREG/TRII Variants in Mice

A single dose of anti-AREG/TRII variants 010, 013, and 030 at 15 mg/mg was administered to C57/B16 mice. Blood samples were collected at pre-dose, 3 h, 8 h, 24 h, 48 h, 72 h, 120 h, 168 h, 336 h, and 504 h after dosing. Serum samples were separated using a standard protocol and then stored below −60° C. until analysis.

The analytic procedure is listed as below:
1) Coating: Add 100 μL of 3 μg/mL Streptavidin (Sigma, S4762) to each well, then seal the plate and incubate it overnight at 2-8° C.
2) Blocking: Discard the contents in the wells, wash each well with 300 μL wash buffer for 3 times, and dry the plate. Add 100 μL of biotinylated AREG-hFc at a concentration of 1 μg/mL in dilution buffer into each well. Seal the plate and incubate it for about 1 hour at 30° C.
3) Add Samples: Discard the contents in the wells, wash each well with 300 μL wash buffer for 3 times, and dry the plate. Serum samples and calibration curve samples are diluted with the dilution buffer. Then, 100 μL of each processed sample is added into each well of the plate. Seal the plate and incubate it for about 1 hour at 30° C.
4) Add Anti-TRII antibody working solution: Discard the contents in the wells. Wash each well with 300 μL wash buffer for 6 times and dry the plate. Add 100 μL of 5 μg/mL anti-TRII antibody working solution into each well of the plate and incubate for about 1 hour at 30° C.
5) Add detection antibody working solution: Discard the contents of in the wells. Wash each well with 300 μL wash buffer for 6 times and dry the plate. Add 100 μL of the detection antibody working solution into each well of the plate and incubate for about 0.5 hour at 30° C.
6) Color development: Discard the contents in the wells. Wash each well with 300 μL wash buffer for 6 times and blot the plate dry. Add 100 μL of the substrate solution into each well. Incubate the plate for 10 min at room temperature and protect it from light.
7) Read plate: Add 50 μL stop solution into each well and read the plate with a plate reader within 30 min at the wavelength of 450 nm with a reference wavelength set at 630 nm.
8) Result output: Plot the calibration standard curve using the calibration concentrations as the X-axis and corresponding absorbance (OD) values (OD450 nm-OD630 nm, without deducting blank) as the Y-axis to calculate concentrations of samples.

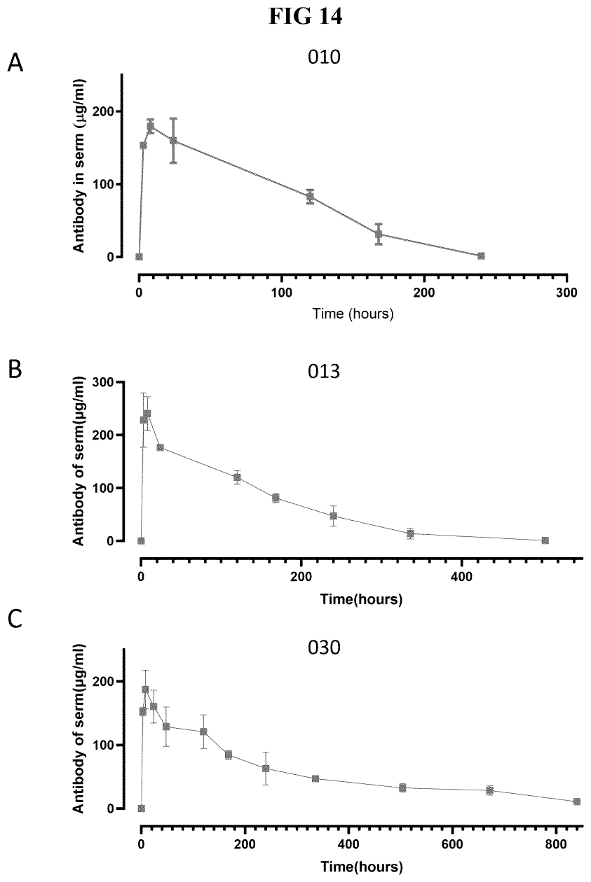

As shown in FIG. 14, after a single dose of intraperitoneal injection of 010, 013, and 030 at 15 mg/kg, these variants exhibited a similar Cmax ranged from 200 to 250 μg/ml and a half-life around 4-10 days.

### Example 13. In-Vivo Efficacy of Anti-AREG/TRII Fusion Protein

In vivo efficacy of the anti-AREG/TRII fusion protein is demonstrated in a progressive lung fibrosis animal model using a surrogate molecule. In this animal model, loss of Cdc42 (encoding CDC42, cell division control protein 42 homolog) in alveolar type II (AT2s) leads to impaired alveolar regeneration and progressive lung fibrosis following the pneumonectomy (PNX)-induced lung injury in mice. Fibrosis in this model, abbreviated as Cdc42 AT2 null model hereafter, is characterized by a periphery-to-center progression of scarring, recapitulating the progression of the disease in IPF patients. Cdc42 AT2 null mice have a significantly decreased body weight and a dramatically reduced survival rate because of the progression of fibrosis in these animals. The anti-AREG/TRII fusion protein contains a light chain (SEQ ID NO: 141) and a heavy chain fused to TRII via a linker (SEQ ID NO: 142), in which the anti-AREG is specifically bind to AREG of the animal model.

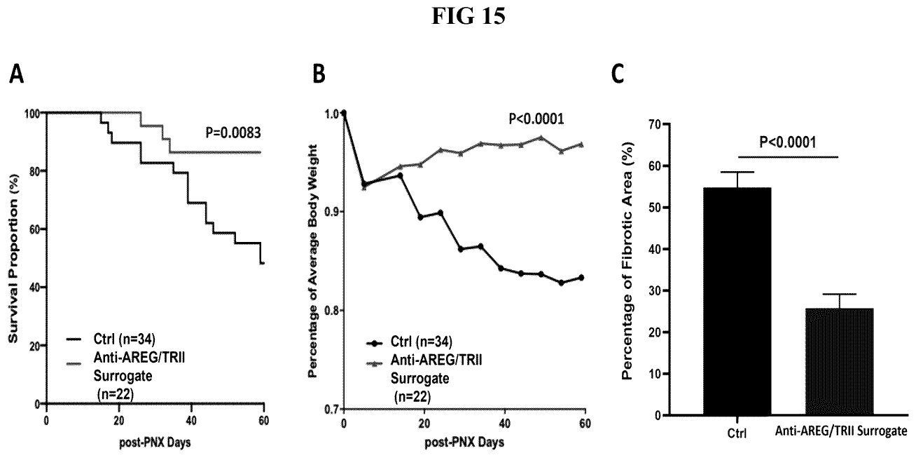

The detailed generation of the mouse fibrosis model has been described previously (WO2020237587A1). Briefly, Cdc42 flox/flox mice were crossed with Spc-CreER-rtTA. Tamoxifen injection was performed to specifically delete Cdc42 in AT2s. These transgenic mice then underwent partial pneumonectomy (PNX) to have their left lung lobes removed to allow for the increased mechanical tension to induce fibrosis. Starting at Day 14 after PNX, Cdc42 AT2 null mice were administered with the anti-AREG/TRII surrogate molecule at the dose 15 mg/kg every 5 days until D60 post-PNX. Body weight was measured every 5 days. Treatment of the surrogate molecule consistently showed efficacy as demonstrated by significantly improved survival (P=0.0083; FIG. 15A), significantly decreased body weight loss (P<0.0001; FIG. 15B), and significantly decreased fibrotic foci and areas as indicated by the fibrosis score (P<0.0001; FIG. 15C). Therefore, anti-AREG/TRII mechanism is sufficient to decrease fibrosis development.

### Discussion

We have shown that the constructed bifunctional anti-AREG/TRII fusion protein can 1) efficiently block the AREG-EGFR signaling in a pEGFR immunoblotting assay and a SRE reporter assay, and 2) efficiently inhibit the TGFβ signaling as demonstrated by preventing pSMAD2 nuclear localization by immunostaining, decreasing phosphorylation of SMAD2 by immunoblotting, and inhibiting the induction of a SBE reporter induced by TGFβ1. Additionally, we show that anti-AREG/TRII variants can simultaneously target AREG and TGFβ signaling. These results together demonstrate that anti-AREG/TRII bi-functional fusion protein is capable of blocking both AREG and TGFβ signaling and could be used as a therapeutic molecule for fibrosis, chronic inflammation, and cancer.

1. A bi-functional fusion protein comprising a first domain and a second domain, wherein
the first domain is capable of binding to AREG or a fragment thereof, preferably is an antibody or an antigen-binding fragment thereof that binds to AREG or a fragment thereof, and
the second domain is capable of binding to a TGFβ ligand or a fragment thereof, preferably comprises a part of the ectodomain of TGFβ receptor II (TRII) or a variant thereof.

2.-3. (canceled)

4. The bi-functional fusion protein of claim 1, wherein the antibody or the antigen-binding fragment thereof is capable of binding to both human AREG and mouse AREG, alternatively, the anti-AREG antibody or the fragment thereof is capable of binding to human AREG with weak or without cross-reactivity to mouse AREG, and/or
wherein the anti-AREG antibody or the fragment thereof is a human anti-AREG antibody, a murine anti-AREG antibody, a chimeric anti-AREG antibody, or a humanized anti-AREG antibody, preferably, is a human monoclonal antibody (mAb), murine mAb, chimeric mAb or humanized mAb, and/or
wherein the anti-AREG antibody or the fragment thereof is capable of binding to a soluble form of AREG, preferably, is capable of binding to an epidermal growth factor (EGF)-like domain of the soluble form of AREG, and/or
wherein the anti-AREG antibody or the fragment thereof is an isotype of IgG, IgM, IgA, IgE, IgD or a variant thereof, preferably, an isotype of IgG1, IgG2, IgG3, IgG4 or a variant thereof.

5.-6. (canceled)

7. The bi-functional fusion protein of claim 1, wherein the anti-AREG antibody or the fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, and HCDR3 are selected from a group consisting of:
(1) HCDR1: SYAMS (SEQ ID NO: 1), HCDR2: AISGSGGSTYYADSVKG (SEQ ID NO: 2), HCDR3: PTSRYSYGYDY (SEQ ID NO: 3);
(2) HCDR1: SYAMS (SEQ ID NO: 1), HCDR2: AISGSGGSTYYADSVKG (SEQ ID NO: 2), HCDR3: PTSRYSYSYNN (SEQ ID NO: 4);
(3) HCDR1: SHAMS (SEQ ID NO: 5), HCDR2: AISGSGGSTYYADSVKG (SEQ ID NO: 2), HCDR3: VDTKFDP (SEQ ID NO: 6);
(4) HCDR1: SYPMS (SEQ ID NO: 7), HCDR2: TISTGGTYTYYPDSVKG (SEQ ID NO: 8), HCDR3: QGPIYYGNYYYAMDY (SEQ ID NO: 9);
(5) HCDR1: SYPMS (SEQ ID NO: 7), HCDR2: TISTGGRYTYYPDSVKG (SEQ ID NO: 10), HCDR3: QGPIYYGNYYYAMDY (SEQ ID NO: 9);
(6) HCDR1: SYPMS (SEQ ID NO: 7), HCDR2: TISTGGTYTYYPDSVKG (SEQ ID NO: 8), HCDR3: QGPILRKNYYYGMDV (SEQ ID NO: 11);
(7) HCDR1: SYPMS (SEQ ID NO: 7), HCDR2: TISTGGTYTYYPDSVKG (SEQ ID NO: 8), HCDR3: QGPIYYGNYYYGMDV (SEQ ID NO: 12);
(8) HCDR1: SYAMS (SEQ ID NO: 1), HCDR2: TISTGGSHTYYPDSVKG (SEQ ID NO: 13), HCDR3: HGYLLYDGYYEWYFDV (SEQ ID NO: 14);
(9) HCDR1: SYAMS (SEQ ID NO: 1), HCDR2: TISTGGSHTYYPDSVKG (SEQ ID NO: 13), HCDR3: HGYLLYDGYYEWYFDY (SEQ ID NO: 140);
(10) HCDR1: SYAMS (SEQ ID NO: 1), HCDR2: TISTGGSHTYYPESVKG (SEQ ID NO: 15), HCDR3: HGYLLYEGYYEWYFDY (SEQ ID NO: 16);
(11) HCDR1: GYPMS (SEQ ID NO: 17), HCDR2: TISTGARHTYYPDSVKG (SEQ ID NO: 18), HCDR3: HEGLRRGKYHCIMDY (SEQ ID NO: 19);
(12) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20); and
(13) HCDR1, HCDR2, HCDR3 as shown in (1)-(12), but at least one of which includes one, two, three, four or five amino acids addition, deletion, conservative amino acid substitution or the combinations thereof; and
LCDR1, LCDR2, and LCDR3 are selected from a group consisting of:
(1) LCDR1: TGNSNNVGDQGAV (SEQ ID NO: 21), LCDR2: RNNNRPS (SEQ ID NO: 22), LCDR3: STWDSGLNSVV (SEQ ID NO: 23);
(2) LCDR1: TGNSNNVGDQGAV (SEQ ID NO: 21), LCDR2: RNNNRPS (SEQ ID NO: 22), LCDR3: STWDKNNKSVV (SEQ ID NO: 24);
(3) LCDR1: SGSSSNIGSNTVN (SEQ ID NO: 25), LCDR2: SNNQRPS (SEQ ID NO: 26), LCDR3: EVWDDSLNGPV (SEQ ID NO: 27);
(4) LCDR1: RSSQSLVHSDGNTYLH (SEQ ID NO: 28), LCDR2: KVSNRFS (SEQ ID NO: 29), LCDR3: SQSTHVPYT (SEQ ID NO: 30);
(5) LCDR1: RSSQSLVDGEDGTYLN (SEQ ID NO: 31), LCDR2: KVSERFD (SEQ ID NO: 32), LCDR3: SQSTHVPYT (SEQ ID NO: 30);
(6) LCDR1: RSSQSLVDGQDGTYLH (SEQ ID NO: 33), LCDR2: KVSNRFD (SEQ ID NO: 34), LCDR3: SQSTHVPYT (SEQ ID NO: 30);
(7) LCDR1: RSSQSLVNQEGETYLH (SEQ ID NO: 35), LCDR2: KVSNRFD (SEQ ID NO: 34), LCDR3: SQSTHVPYT (SEQ ID NO: 30);
(8) LCDR1: KASQSVDYDGHSFLN (SEQ ID NO: 36), LCDR2: AASNLES (SEQ ID NO: 37), LCDR3: QQSTEDPPYT (SEQ ID NO: 38);
(9) LCDR1: RASESVDYDGHSFIN (SEQ ID NO: 39), LCDR2: AASNKDT (SEQ ID NO: 40), LCDR3: QQSTEDPPYT (SEQ ID NO: 38);
(10) LCDR1: RASQSVDYDGHSFLN (SEQ ID NO: 41), LCDR2: AASNLQS (SEQ ID NO: 42), LCDR3: QQSTEDPPYT (SEQ ID NO: 38);
(11) LCDR1: KSSQSVDYDGHSFLN (SEQ ID NO: 43), LCDR2: AASNRES (SEQ ID NO: 44), LCDR3: QQSTEDPPYT (SEQ ID NO: 38);
(12) LCDR1: RASESVDYDGHSFIN (SEQ ID NO: 39), LCDR2: AASNKDT (SEQ ID NO: 40), LCDR3: QQSTEDPPYT (SEQ ID NO: 38);
(13) LCDR1: RASQSVDYEGHSFLN (SEQ ID NO: 45), LCDR2: AASNLQS (SEQ ID NO: 42), LCDR3: QQSTENPPYT (SEQ ID NO: 46);
(14) LCDR1: KSSQSVDYEGHSFLN (SEQ ID NO: 47), LCDR2: AASNRES (SEQ ID NO: 44), LCDR3: QQSTENPPYT (SEQ ID NO: 46);
(15) LCDR1: KASQSIDYDGDSFLN (SEQ ID NO: 48), LCDR2: AASNLES (SEQ ID NO: 37), LCDR3: HQCNEDPYM (SEQ ID NO: 49);
(16) LCDR1: RASESVDYDGDSFIN (SEQ ID NO: 50), LCDR2: AASNKDT (SEQ ID NO: 40), LCDR3: HQSNEDPYM (SEQ ID NO: 51);
(17) LCDR1: RASESVDYDGDSFIN (SEQ ID NO: 50), LCDR2: AASNKDT (SEQ ID NO: 40), LCDR3: HQSNEDPYL (SEQ ID NO: 52);
(18) LCDR1: RASESVDYDGDSFIN (SEQ ID NO: 50), LCDR2: AASNKDT (SEQ ID NO: 40), LCDR3: HQSNEDPYV (SEQ ID NO: 53);
(19) LCDR1: RASQSIDYDGDSFLN (SEQ ID NO: 54), LCDR2: AASNLQS (SEQ ID NO: 42), LCDR3: QQSNEDPYV (SEQ ID NO: 55);
(20) LCDR1: KSSQSIDYDGDSFLN (SEQ ID NO: 56), LCDR2: AASNRES (SEQ ID NO: 44), LCDR3: QQSNEDPYV (SEQ ID NO: 55); and
(21) LCDR1, LCDR2, LCDR3 as shown in (1)-(20), but at least one of which includes one, two, three, four or five amino acids addition, deletion, conservative amino acid substitution or the combinations thereof.

8. The bi-functional fusion protein of claim 1, wherein the anti-AREG antibody or a fragment thereof comprises a heavy chain variable region comprising heavy chain complementarity determining regions HCDR1, HCDR2, and HCDR3, and a light chain variable region comprising light chain complementarity determining regions LCDR1, LCDR2, and LCDR3, wherein:
HCDR1, HCDR2, HCDR3, LCDR1, LCDR2, and LCDR3 are selected from a group consisting of:
(1) HCDR1: SYAMS (SEQ ID NO: 1), HCDR2: AISGSGGSTYYADSVKG (SEQ ID NO: 2), HCDR3: PTSRYSYGYDY (SEQ ID NO: 3), LCDR1 TGNSNNVGDQGAV (SEQ ID NO: 21), LCDR2: RNNNRPS (SEQ ID NO: 22), LCDR3: STWDSGLNSVV (SEQ ID NO: 23);
(2) HCDR1: SYAMS (SEQ ID NO: 1), HCDR2: AISGSGGSTYYADSVKG (SEQ ID NO: 2), HCDR3: PTSRYSYSYNN (SEQ ID NO: 4), LCDR1: TGNSNNVGDQGAV (SEQ ID NO: 21), LCDR2: RNNNRPS (SEQ ID NO: 22), LCDR3: STWDKNNKSVV (SEQ ID NO: 24);
(3) HCDR1: SHAMS (SEQ ID NO: 5), HCDR2: AISGSGGSTYYADSVKG (SEQ ID NO: 2), HCDR3: VDTKFDP (SEQ ID NO: 6), LCDR1: SGSSSNIGSNTVN (SEQ ID NO: 25), LCDR2: SNNQRPS (SEQ ID NO: 26), LCDR3: EVWDDSLNGPV (SEQ ID NO: 27);
(4) HCDR1: SYPMS (SEQ ID NO: 7), HCDR2: TISTGGTYTYYPDSVKG (SEQ ID NO: 8), HCDR3: QGPIYYGNYYYAMDY (SEQ ID NO: 9), LCDR1: RSSQSLVHSDGNTYLH (SEQ ID NO: 28), LCDR2: KVSNRFS (SEQ ID NO: 29), LCDR3: SQSTHVPYT (SEQ ID NO: 30);
(5) HCDR1: SYPMS (SEQ ID NO: 7), HCDR2: TISTGGRYTYYPDSVKG (SEQ ID NO: 10), HCDR3: QGPIYYGNYYYAMDY (SEQ ID NO: 9), LCDR1: RSSQSLVDGEDGTYLN (SEQ ID NO: 31), LCDR2: KVSERFD (SEQ ID NO: 32), LCDR3: SQSTHVPYT (SEQ ID NO: 30);
(6) HCDR1: SYPMS (SEQ ID NO: 7), HCDR2: TISTGGTYTYYPDSVKG (SEQ ID NO: 8), HCDR3: QGPILRKNYYYGMDV (SEQ ID NO: 11), LCDR1: RSSQSLVDGQDGTYLH (SEQ ID NO: 33), LCDR2: KVSNRFD (SEQ ID NO: 34), LCDR3: SQSTHVPYT (SEQ ID NO: 30);
(7) HCDR1: SYPMS (SEQ ID NO: 7), HCDR2: TISTGGTYTYYPDSVKG (SEQ ID NO: 8), HCDR3: QGPIYYGNYYYGMDV (SEQ ID NO: 12), LCDR1: RSSQSLVNQEGETYLH (SEQ ID NO: 35), LCDR2: KVSNRFD (SEQ ID NO: 34), LCDR3: SQSTHVPYT (SEQ ID NO: 30);
(8) HCDR1: SYAMS (SEQ ID NO: 1), HCDR2: TISTGGSHTYYPDSVKG (SEQ ID NO: 13), HCDR3: HGYLLYDGYYEWYFDV (SEQ ID NO: 14), LCDR1: KASQSVDYDGHSFLN (SEQ ID NO: 36), LCDR2: AASNLES (SEQ ID NO: 37), LCDR3: QQSTEDPPYT (SEQ ID NO: 38);
(9) HCDR1: SYAMS (SEQ ID NO: 1), HCDR2: TISTGGSHTYYPDSVKG (SEQ ID NO: 13), HCDR3: HGYLLYDGYYEWYFDY (SEQ ID NO: 140), LCDR1: RASESVDYDGHSFIN (SEQ ID NO: 39), LCDR2: AASNKDT (SEQ ID NO: 40), LCDR3: QQSTEDPPYT (SEQ ID NO: 38);
(10) HCDR1: SYAMS (SEQ ID NO: 1), HCDR2: TISTGGSHTYYPDSVKG (SEQ ID NO: 13), HCDR3: HGYLLYDGYYEWYFDY (SEQ ID NO: 140), LCDR1: RASQSVDYDGHSFLN (SEQ ID NO: 41), LCDR2: AASNLQS (SEQ ID NO: 42), LCDR3: QQSTEDPPYT (SEQ ID NO: 38);
(11) HCDR1: SYAMS (SEQ ID NO: 1), HCDR2: TISTGGSHTYYPDSVKG (SEQ ID NO: 13), HCDR3: HGYLLYDGYYEWYFDY(SEQ ID NO: 140), LCDR1: KSSQSVDYDGHSFLN (SEQ ID NO: 43), LCDR2: AASNRES (SEQ ID NO: 44), LCDR3: QQSTEDPPYT (SEQ ID NO: 38);
(12) HCDR1: SYAMS (SEQ ID NO: 1), HCDR2: TISTGGSHTYYPESVKG (SEQ ID NO: 15), HCDR3: HGYLLYEGYYEWYFDY (SEQ ID NO: 16), LCDR1: RASESVDYDGHSFIN (SEQ ID NO: 39), LCDR2: AASNKDT (SEQ ID NO: 40), LCDR3: QQSTEDPPYT (SEQ ID NO: 38);
(13) HCDR1: SYAMS (SEQ ID NO: 1), HCDR2: TISTGGSHTYYPESVKG (SEQ ID NO: 15), HCDR3: HGYLLYEGYYEWYFDY (SEQ ID NO: 16), LCDR1: RASQSVDYEGHSFLN (SEQ ID NO: 45), LCDR2: AASNLQS (SEQ ID NO: 42), LCDR3: QQSTENPPYT (SEQ ID NO: 46);
(14) HCDR1: SYAMS (SEQ ID NO: 1), HCDR2: TISTGGSHTYYPESVKG (SEQ ID NO: 15), HCDR3: HGYLLYEGYYEWYFDY (SEQ ID NO: 16), LCDR1: KSSQSVDYEGHSFLN (SEQ ID NO: 47), LCDR2: AASNRES (SEQ ID NO: 44), LCDR3: QQSTENPPYT (SEQ ID NO: 46);
(15) HCDR1: GYPMS (SEQ ID NO: 17), HCDR2: TISTGARHTYYPDSVKG (SEQ ID NO: 18), HCDR3: HEGLRRGKYHCIMDY (SEQ ID NO: 19), LCDR1: KASQSIDYDGDSFLN (SEQ ID NO: 48), LCDR2: AASNLES (SEQ ID NO: 37), LCDR3: HQCNEDPYM (SEQ ID NO: 49);
(16) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20), LCDR1: RASESVDYDGDSFIN (SEQ ID NO: 50), LCDR2: AASNKDT (SEQ ID NO: 40), LCDR3: HQSNEDPYM (SEQ ID NO: 51);
(17) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20), LCDR1: RASESVDYDGDSFIN (SEQ ID NO: 50), LCDR2: AASNKDT (SEQ ID NO: 40), LCDR3: HQSNEDPYL (SEQ ID NO: 52);
(18) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20), LCDR1: RASESVDYDGDSFIN (SEQ ID NO: 50), LCDR2: AASNKDT (SEQ ID NO: 40), LCDR3: HQSNEDPYV (SEQ ID NO: 53);
(19) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20), LCDR1: RASQSIDYDGDSFLN (SEQ ID NO: 54), LCDR2: AASNLQS (SEQ ID NO: 42), LCDR3: QQSNEDPYV (SEQ ID NO: 55);
(20) HCDR1: GYPMS (SEQ ID NO: 17), HCDR2: TISTGARHTYYPDSVKG (SEQ ID NO: 18), HCDR3: HEGLRRGKYHSIMDY (SEQ ID NO: 20), LCDR1: KSSQSIDYDGDSFLN (SEQ ID NO: 56), LCDR2: AASNRES (SEQ ID NO: 44), LCDR3: QQSNEDPYV (SEQ ID NO: 55); and
(21) HCDR1, HCDR2, HCDR3, LCDR1, LCDR2, LCDR3 as shown in (1)-(20), but at least one of which includes one, two, three, four or five amino acids addition, deletion, conservative amino acid substitution or the combinations thereof.

9. The bi-functional fusion protein of claim 1, wherein the anti-AREG antibody or the fragment thereof comprises a heavy chain variable region, and a light chain variable region, wherein the heavy chain variable region has an amino acid sequence selected from a group consisting of SEQ ID NOs: 57-69, and an amino acid sequence having at least 95% sequence identity to any one of SEQ ID NOs: 57-69, and retaining epitope-binding activity,
wherein the light chain variable region has an amino acid sequence selected from a group consisting of SEQ ID NOs: 70-89, and an amino acid sequence having at least 95% sequence identity to any one of SEQ ID NOs: 70-89, and retaining epitope-binding activity.

10. The bi-functional fusion protein of claim 1, wherein the anti-AREG antibody or the fragment thereof comprises a heavy chain variable region, and a light chain variable region, wherein the heavy chain variable region and the light chain variable region have an amino acid sequences selected from the group consisting of:
(1) SEQ ID NO: 57 and SEQ ID NO: 70;
(2) SEQ ID NO: 58 and SEQ ID NO: 71;
(3) SEQ ID NO: 59 and SEQ ID NO: 72;
(4) SEQ ID NO: 60 and SEQ ID NO: 73;
(5) SEQ ID NO: 61 and SEQ ID NO: 74;
(6) SEQ ID NO: 62 and SEQ ID NO: 75;
(7) SEQ ID NO: 63 and SEQ ID NO: 76;
(8) SEQ ID NO: 64 and SEQ ID NO: 77;
(9) SEQ ID NO: 65 and SEQ ID NO: 78;
(10) SEQ ID NO: 66 and SEQ ID NO: 79;
(11) SEQ ID NO: 66 and SEQ ID NO: 80;
(12) SEQ ID NO: 66 and SEQ ID NO: 81;
(13) SEQ ID NO: 67 and SEQ ID NO: 79;
(14) SEQ ID NO: 67 and SEQ ID NO: 82;
(15) SEQ ID NO: 67 and SEQ ID NO: 83;
(16) SEQ ID NO: 68 and SEQ ID NO: 84;
(17) SEQ ID NO: 69 and SEQ ID NO: 85;
(18) SEQ ID NO: 69 and SEQ ID NO: 86;
(19) SEQ ID NO: 69 and SEQ ID NO: 87;
(20) SEQ ID NO: 69 and SEQ ID NO: 88;
(21) SEQ ID NO: 69 and SEQ ID NO: 89; and
(22) two amino acid sequences having at least 95% sequence identity to any one of (1)-(21) respectively, and retaining epitope-binding activity.

11. (canceled)

12. The bi-functional fusion protein of claim 1, wherein the second domain comprises the ectodomain of TRII or a variant thereof, preferably the variant comprises a site mutation, and/or a deletion,
preferably the ectodomain of TRII has an amino acid sequence shown in SEQ ID NO: 90, or an amino acid sequence that is at least 85% identical to SEQ ID NO: 90.

13-14. (canceled)

15. The bi-functional fusion protein of claim 12, wherein the site mutation comprises one or more site mutations at position(s) selected from K7, T16, D17, R34, R66, K67, K103, and K104 on the basis of the numbering of SEQ ID NO: 90 from N-terminus to C-terminus,
preferably the site mutation comprises one or more site mutations selected from K7Q, T16S, D17N, R34S, R34H, R66S, K67S, K103S, and K104S on the basis of the numbering of SEQ ID NO: 90 from N-terminus to C-terminus, more preferably the site mutation is selected from: T16S and D17N: K7Q and D17N: K7Q;
R34S; R34H: R66S and K67S; K103S and K104S; K7Q and R34S; K7Q, R66S and K67S; K7Q, K103S, and K104S; K7Q, R34S, R66S, and K67S; K7Q, R34S, K103S, and K104S; K7Q, R66S, K67S, K103S, and K104S; R34S, R66S, K67S, K103S, and K104S; K7Q, R34S, R66S, K67S, K103S, and K104S.

16.-17. (canceled)

18. The bi-functional fusion protein of claim 12, wherein the variant has an N-terminus deletion of four to twenty-one amino acids, preferably, four amino acids, seven amino acids, nine amino acids, thirteen amino acids, seventeen amino acids, and twenty-one amino acids, on the basis of the numbering of SEQ ID NO: 90 from N-terminus to C-terminus.

19. The bi-functional fusion protein of claim 12, wherein the variant comprises site mutations T16S and D17N, and an N-terminus deletion of seven amino acids.

20. The bi-functional fusion protein of claim 12, wherein the second domain comprises the ectodomain of TRII or a variant thereof and has an amino acid sequence as shown in any one of SEQ ID NOs: 90-107, or an amino acid sequence having at least 85% identity to any one of SEQ ID NOs: 90-107.

21. (canceled)

22. The bi-functional fusion protein of claim 1, wherein C-terminus of a heavy chain or a light chain of the anti-AREG antibody is fused with N-terminus of the ectodomain of TRII or its variant directly, or via a linker; or N-terminus of a heavy chain or a light chain of the anti-AREG antibody is connected with C-terminus of the ectodomain of TRII or its variant directly, or via a linker,
preferably the bi-functional fusion protein comprises: a heavy chain of the anti-AREG antibody, at its N-terminus, connected with C-terminus of the ectodomain of TRII or its variant directly, or via a linker; or, a heavy chain of the anti-AREG antibody, at its C-terminus, connected with N-terminus of the ectodomain of TRII or its variant directly, or via a linker,
preferably the bi-functional fusion protein further comprises a light chain of the anti-AREG antibody, and/or the bi-functional fusion protein is in a form of heterotetramer.

23.-24. (canceled)

25. The bi-functional fusion protein of claim 22, wherein the linker comprises a linker peptide shown by a formula (G₄S)ₙ, (G₄S)ₙG, S(G₄S)ₙG, SG(EAAAK)ₙSG, S(GEGES)ₙG, or (EAAAK)ₙ, wherein n is an integer of 1 to 5, preferably, the linker peptide has an amino acid sequence shown by any one of SEQ ID NOs: 108-117.

26. The bi-functional fusion protein of claim 1, wherein the bi-functional fusion protein comprises a heavy chain of the anti-AREG antibody, at its C-terminus, connected with N-terminus of the ectodomain of TRII via a linker, and has an amino acid sequence shown in any one of SEQ ID NOs: 118-139 and 142, or an amino acid sequence having at least 85% identity to any one of SEQ ID NOs: 118-139 and 142.

27.-28. (canceled)

29. An isolated nucleic acid molecule, encoding the bi-functional fusion protein as defined in claim 1.

30. An expression vector, comprising the isolated nucleic acid molecule of claim 29.

31. A host cell, which comprises the isolated nucleic acid molecule of claim 29.

32. (canceled)

33. A pharmaceutical composition, comprising the bi-functional fusion protein of claim 1 and a pharmaceutically acceptable carrier.

34. (canceled)

35. A method for preventing, treating and/or diagnosing fibrotic diseases, cancers and diseases associated with chronic inflammation in a subject, which comprises administering to a subject a therapeutically effective amount of the bi-functional fusion protein of claim 1, preferably, the fibrotic diseases include renal fibrosis, hepatic fibrosis, pulmonary fibrosis, in particular, idiopathic pulmonary fibrosis (IPF).

36. A method for preventing, treating and/or diagnosing fibrotic diseases, cancers and diseases associated with chronic inflammation in a subject, which comprises administering to a subject a therapeutically effective amount of the pharmaceutical composition of claim 33, preferably, the fibrotic diseases include renal fibrosis, hepatic fibrosis, pulmonary fibrosis, in particular, idiopathic pulmonary fibrosis (IPF).
